(19) United States
(12) Patent Application Publication
(10) Pub. No.: US 2018/0047177 A1
(43) Pub. Date: Feb. 15, 2018

(54) SYSTEMS, DEVICES, AND METHODS FOR MONITORING AND ASSESSING CHARACTERISTICS OF HARVESTED SPECIALTY CROPS

(71) Applicant: Raptor Maps, Inc., Somerville, MA (US)

(72) Inventors: Edward Obropta, Cambridge, MA (US); Mike Klinker, Cambridge, MA (US); Nikhil Vadhavkar, Cambridge, MA (US)

(73) Assignee: Raptor Maps, Inc., Somerville, MA (US)

(21) Appl. No.: 15/677,419

(22) Filed: Aug. 15, 2017

Related U.S. Application Data
(60) Provisional application No. 62/375,365, filed on Aug. 15, 2016.

(51) Int. Cl. G06T 7/62; G06T 7/00; G06T 17/20; G06T 7/13; G01N 33/00; G01B 11/24

(52) U.S. Cl. CPC G06T 7/62; G01N 33/0098; G01B 11/24; G06T 17/20; G06T 7/13; G06T 7/001; G06T 2207/10028; G06T 2207/30188; G06T 2207/20081; G06T 2207/30128

(57) ABSTRACT

A system for use in connection with assessing characteristics of harvested specialty crops, the system comprising: at least one computer hardware processor; and at least one non-transitory computer-readable storage medium storing processor-executable instructions that, when executed by the at least one computer hardware processor, cause the at least one computer hardware processor to perform: obtaining an image of a set of harvested specialty crops and associated depth information; generating a 3D surface model of the set of harvested specialty crops using the image and the depth information; and estimating the volume of the set of harvested specialty crops using the 3D surface model.

### CROSS-REFERENCE TO RELATED APPLICATIONS

This application claims the benefit under 35 U.S.C. § 119(e) of U.S. Provisional Patent Application Ser. No. 62/375,365, filed Aug. 15, 2016, and entitled “HARVEST MONITORING SYSTEM FOR LOCATION-BASED MEASUREMENT OF CROP QUALITY AND QUANTITY,” the entire contents of which is incorporated by reference herein.

### FIELD

The techniques described herein are directed generally to the field of measuring crop yield, and more particularly to techniques for using sensors to measure the quality and/or quantity of harvested crops.

### BACKGROUND

Specialty crops include fruits and vegetables, tree nuts, dried fruits, horticulture, and nursery crops including floriculture. Examples of specialty crops include potatoes, carrots, bell peppers, onions, tomatoes, oranges, and/or any crop indicated by the United States Department of Agriculture (USDA) to be a specialty crop. Examples of crops that are not specialty crops include oil seed crops (e.g., sunflower seeds, sesame, peanuts and soybeans), grain crops (e.g., buckwheat, oats, rye, and wheat), forage crops (e.g., alfalfa, clover, and hay) and fiber crops (e.g., cotton, flax, and hemp).

Crop yield refers to a quantitative measure of the quantity of crops harvested. For example, crop yield may be measured as crop weight per unit area. Generally, the greater the crop yield, the more crops a farmer has produced and the more compensation he or she is likely to receive. However, the farmer's compensation may also depend on crop quality. For specialty crops, the crop quality may be determined by grading of individual specialty crops based on one or more factors including, but not limited to, size, shape, exterior color, interior color, virus, disease, bruising, density, firmness, abrasions, scratch, graze, rupture, bleaching, greening, rot, decay, rough spots, sprouting, lesions, and wilting. Crop quality may also depend on chemical properties of the crops including, but not limited to, amino acid content, nitrate content, salt (e.g., sodium or potassium) content, starch content, and sugar content.

### SUMMARY

Some embodiments provide for a device for use in connection with monitoring and assessing characteristics of harvested specialty crops. The device comprises: an imaging sensor configured to capture an image of a set of harvested specialty crops; a depth sensor; processing circuitry configured to generate depth information at least in part by using data obtained by the depth sensor; and a transmitter configured to transmit the image and the depth information to at least one remote computing device via a communication network.

Some embodiments provide for a system, comprising: specialty crop harvesting equipment (e.g., a harvester, a bin piler, 10-wheel trucks, conveyor, etc.), the specialty crop harvesting equipment including a conveyor; and a device coupled to the specialty crop harvesting equipment, the device comprising: an imaging sensor configured to capture an image of harvested specialty crops on the conveyor; a depth sensor; processing circuitry configured to generate depth information at least in part by using data obtained by the depth sensor; and a transmitter configured to transmit the image and the depth information to at least one remote computing device via a communication network.

Some embodiments provide for a method for use in connection with monitoring and assessing characteristics of harvested specialty crops. The method comprises using at least one computer hardware processor to perform: obtaining an image of a set of harvested specialty crops, the image obtained using an imaging sensor; obtaining depth data obtained using a depth sensor; generating depth information using the depth data; and transmitting, via a communication network, the image and the depth information to at least one remote computing device.

Some embodiments provide for at least one non-transitory computer-readable storage medium storing processor-executable instructions that, when executed by at least one computer hardware processor (e.g., part of a device coupled to a specialty crop harvester, a bin piler, etc.), cause the at least one computer hardware processor to perform a method for use in connection with monitoring and assessing characteristics of harvested specialty crops. The method comprises using at least one computer hardware processor to perform: obtaining an image of a set of harvested specialty crops, the image obtained using an imaging sensor; obtaining depth data obtained using a depth sensor; generating depth information using the depth data; and transmitting, via a communication network, the image and the depth information to at least one remote computing device.

Some embodiments provide for a system for use in connection with assessing characteristics of harvested specialty crops. The system comprises: at least one computer hardware processor; and at least one non-transitory computer-readable storage medium storing processor-executable instructions that, when executed by the at least one computer hardware processor, cause the at least one computer hardware processor to perform: obtaining an image of a set of harvested specialty crops and associated depth information; generating a 3D surface model of the set of harvested specialty crops using the image and the depth information; and estimating the volume of the set of harvested specialty crops using the 3D surface model.

Some embodiments provide for a method for use in connection with assessing characteristics of harvested specialty crops. The method comprises: obtaining an image of a set of harvested specialty crops and associated depth information; generating a 3D surface model of the set of harvested specialty crops using the image and the depth information; and estimating the volume of the set of harvested specialty crops using the 3D surface model.

Some embodiments provide for at least one non-transitory computer-readable storage medium programmed with processor-executable instructions that, when executed by at least one computer hardware processor, cause the at least one computer hardware processor to perform a method for use in connection with assessing characteristics of harvested specialty crops. The method comprises: obtaining an image of a set of harvested specialty crops and associated depth information; generating a 3D surface model of the set of harvested specialty crops using the image and the depth information; and estimating the volume of the set of harvested specialty crops using the 3D surface model.

Some embodiments provide for a system for use in connection with assessing characteristics of harvested specialty crops. The system comprises at least one computer hardware processor; and at least one non-transitory computer-readable storage medium storing processor-executable instructions that, when executed by the at least one computer hardware processor, cause the at least one computer hardware processor to perform: obtaining an image of a set of harvested specialty crops and associated depth information; and determining, using the image and the depth information, the size and/or shape of each of multiple specialty crops in the set of harvested specialty crops.

Some embodiments provide for a method for use in connection with assessing characteristics of harvested specialty crops. The method comprises using at least one computer hardware processor to perform: obtaining an image of a set of harvested specialty crops and associated depth information; and determining, using the image and the depth information, the size and/or shape of each of multiple specialty crops in the set of harvested specialty crops.

Some embodiments provide for at least one non-transitory computer-readable storage medium programmed with processor-executable instructions that, when executed by at least one computer hardware processor, cause the at least one computer hardware processor to perform a method for use in connection with assessing characteristics of harvested specialty crops. The method comprises using at least one computer hardware processor to perform: obtaining an image of a set of harvested specialty crops and associated depth information; and determining, using the image and the depth information, the size and/or shape of each of multiple specialty crops in the set of harvested specialty crops.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

The accompanying drawings are not intended to be drawn to scale. For purposes of clarity, not every component may be labeled in every drawing. In the drawings:

FIG. 1A shows an illustrative environment in which some embodiments of the technology described herein may operate.

FIG. 1B shows a block diagram of an illustrative device comprising a plurality of sensors (sometimes referred to as a “sensor package” herein) for use in connection with monitoring and/or assessing characteristics of harvested specialty crops, in accordance with some embodiments of the technology described herein.

FIG. 1C shows a block diagram of another illustrative device comprising a plurality of sensors for use in connection with monitoring and/or assessing characteristics of harvested specialty crops, in accordance with some embodiments of the technology described herein.

FIG. 2A shows illustrative arrangements of one or more sensor packages in a field environment in which some embodiments of the technology described herein may operate.

FIG. 2B shows another illustrative arrangement of a sensor package in a field environment in which some embodiments of the technology described herein may operate.

FIG. 3 shows a sensor package in an illustrative configuration for monitoring harvested specialty crops on a conveyor belt, in accordance with some embodiments of the technology described herein.

FIG. 4 shows an illustrative image of crops being measured for individual characteristics, in accordance with some embodiments of the technology described herein.

FIG. 5 shows an illustrative image of the depth of crops being measured, in accordance with some embodiments of the technology described herein.

FIG. 6 illustrates a three-dimensional surface model of a set of harvested specialty crops, in accordance with some embodiments of the technology described herein.

FIG. 7 is a flow chart of an illustrative process for obtaining and transmitting data for monitoring and assessing characteristics of harvested specialty crops, in accordance with some embodiments of the technology described herein.

FIG. 8 is flow chart of an illustrative process for monitoring and assessing characteristics of harvested specialty crops, in accordance with some embodiments of the technology described herein.

FIG. 9 is a block diagram of an illustrative computer system that may be used in implementing some embodiments of the technology described herein.

### DETAILED DESCRIPTION

The inventors have recognized and appreciated that conventional technology for monitoring specialty crops may be improved upon. First, conventional specialty crop monitoring technology cannot be used to accurately estimate the mass and/or volume of the specialty crops that have been harvested. For example, a conventional in-field monitoring system uses load cells installed under conveyor belts of harvesters to determine the weight of crops being moved by the conveyor belt. However, such a weight-based system cannot distinguish between crops and weeds, rocks, and other debris, all of which is moved by the harvester's conveyor. As a result, such a weight-based system provides poor estimates of the mass of specialty crops harvested. Second, conventional specialty crop monitoring technology cannot be used for obtaining accurate information about size and/or quality of individual specialty crops, which is important for a number of reasons including how much a grower is to be compensated for the harvested specialty crops. For example, conventional weight-based monitoring systems cannot determine whether 100 smaller 5 oz potatoes or 50 larger 10 oz potatoes passed above the scale on the conveyor belt. As another example, although quality may be determined through manual examination, such hand grading techniques do not scale and require “sampling” the harvested crops in that only a small subset of the harvested crops is used to estimate the size and/or quality of the harvest across truckloads and/or fields.

The inventors have also recognized and appreciated that conventional monitoring systems deployed in pack houses are laboratory grade and will not operate in the dirty operational field environment of harvesting equipment. As a result, such conventional monitoring systems are unable to determine the location of harvested specialty crops having given observed size, shape, and/or quality characteristics.

The inventors have also recognized and appreciated that conventional systems for monitoring harvested grain crops are unsuitable for use in monitoring harvested specialty crops. For example, conventional grain yield monitoring systems rely on the homogeneity of grain crops, such that yield can be determined by measuring only the moisture and mass of the grains. Specialty crops exhibit much greater variations in characteristics (e.g., size and/or shape, rot, color, and bruising), however, and neither their mass nor quality can be measured using conventional systems for monitoring harvested grain crops. For example, grain measuring techniques cannot be used to accurately determine yield, quality, individual shape and/or volume of a crop, and many other characteristics that may be useful for the commercialization and cultivation of specialty crops.

Some embodiments of the technology described herein address some of the above-discussed drawbacks of conventional technology for monitoring harvested specialty crops. However, not every embodiment addresses every one of these drawbacks, and some embodiments may not address any of them. As such, it should be appreciated that aspects of the technology described herein are not limited to addressing all or any of the above discussed drawbacks of conventional harvested specialty crops monitoring technology.

Accordingly, some embodiments provide for techniques for monitoring and assessing characteristics of harvested specialty crops. The techniques by the inventors allow for: (1) in-field monitoring of the harvest of specialty crops; (2) measurement of specialty crop yield; (3) determination of the quality and/or size of individual specialty crops; and/or (4) associating any of the measured characteristics of specialty crops with the location(s) of where these specialty crops are harvested and/or stored.

Accordingly, some embodiments provide for a device for use in connection with monitoring and assessing characteristics of harvested specialty crops. In some embodiments, the device may include: (1) an imaging sensor (e.g., a color camera, a monochrome camera, a multi-spectral camera, etc.) configured to capture one or more image(s) of a set of harvested specialty crops; (2) a depth sensor (e.g., an ultrasonic sensor, a LIDAR (Light Detection and Ranging) sensor, another imaging sensor, etc.); (3) processing circuitry configured to generate depth information at least in part by using data obtained by the depth sensor (and, in some embodiments, by also using the image(s) obtained by the imaging sensor); and (4) a transmitter (e.g., a wireless transmitter) configured to transmit the image(s) and the depth information to at least one remote computing device (e.g., a remote server, one or more computing nodes in a cloud computing environment, etc.) via a communication network. The device, sometimes referred to as a “sensor package” herein, contains sensors that collect data used for assessing one or more various characteristics of the harvested specialty crops. In some embodiments, the depth information allows for the creation of a three-dimensional (3D) profile of harvested crops, even if they are being harvested in a single layer.

In some embodiments, depth information generated by the processing circuitry may include information indicating the depth of one or more specialty crops. For example, in some embodiments, depth information may indicate, for a particular specialty crop, the distance between a surface that supports the particular specialty crop and a point on top of the specialty crop (e.g., in a direction normal to the surface). As another example, the depth information may indicate, for a particular specialty crop, the distance between a point on the specialty crop (e.g., on a top surface of the specialty crop) and the sensor package. Such information, together with a known distance between the sensor package and the surface supporting the specialty crop, may be used to determine the depth of the specialty crop.

In some embodiments, the device may also include a location sensor (e.g., a GPS receiver, an accelerometer, a gyroscope, a magnetometer, and/or inertial measurement unit). In such embodiments, the transmitter may be further configured to transmit location data obtained by using the location sensor (or any information derived therefrom) to the at least one remote computing device via the communication network. For example, the location data may indicate a location where the image of a set of specialty crops was captured and/or a location where the imaged specialty crops were harvested. This allows the characteristics (e.g., crop yield and crop quality) of the harvested specialty crops to be associated with information indicating the field locations where the specialty crops were harvested.

In some embodiments, the data obtained by the depth sensor includes multiple distances between the depth sensor (or between the device in which the depth sensor is housed) and the set of harvested specialty crops. For example, the depth sensor may measure a distance between the sensor and the highest point of the harvested specialty crop or a point above a detected centroid of the specialty crop. For example, the depth sensor may measure multiple distances between the depth sensor and the surface of a harvested specialty crop (e.g., by generating a LIDAR point cloud when the depth sensor is a LIDAR sensor or a stereoscopic image when the depth sensor is an imaging device).

In some embodiments, the processing circuitry is configured to compute depths between the harvested crops and the conveyor based on the plurality of distances between the depth sensor and the harvested specialty crops. For example, the processing circuitry may subtract the vertical (e.g., normal to the conveyor) component of the measured distance from a known height between the depth sensor and the conveyor holding the harvested specialty crops. These depths may be used for creating a 3D model of the volume of the flow of harvested specialty crops (e.g., along a conveyor).

In some embodiments, the processing circuitry is configured to generate the depth information using the image captured by the imaging sensor and a second image captured by the second imaging sensor concurrently with the imaging sensor capturing the image. For example, the processing circuitry may provide the image captured by the imaging sensor and the second image captured by the second imaging sensor as input to one or more stereoscopic vision algorithms.

In some embodiments, the device further includes a light source for illuminating the set of harvested specialty crops. For example, a flashlight, flood lamp, LEDs or any suitable light source may shine on the harvested specialty crops in dark conditions to allow for visible spectrum imaging. In this way, information about the yield and/or quality of specialty crops may be obtained even for those specialty crops being harvested at night or other low-light conditions.

In some embodiments, the device includes a mount configured to couple the device to a specialty crop harvester. For example the device may be configured to captures images of the harvested specialty crops as they are moved by a conveyor on the crop harvester or a bin piler.

In some embodiments, the device includes a rechargeable battery configured to recharge using power from the specialty crop harvester. For example, the rechargeable battery may operate as an uninterruptable power supply that allows the processing circuitry to complete operations and shut down after losing a supply of power from the harvester.

In some embodiments, the processing circuitry may be configured to turn on at least one of the imaging sensor, the depth sensor, and the transceiver in response to receiving an indication that the conveyor is in motion. The indication may be provided in any suitable way. For example, the indication may be provided by harvesting equipment (e.g., harvester, bin piler, etc.) to which the device is coupled, by a human user, by a motion sensor part of the device, and/or in any other suitable way, as aspects of the technology described herein are not limited in this respect. This ensures that the sensor package will be operational during harvesting.

In some embodiments, the device includes a thermal imaging sensor. The thermal imaging sensor may be used for any suitable purpose. For example, the thermal imaging sensor may be used to detect thermal radiation from the harvested specialty crops. Such information may be used to determine whether the temperature of the specialty crops is suitable for harvesting. Harvesting crops whose temperature is too high may not be desirable, for example. Including a thermal imaging sensor in the sensor package allows for the sensor package, in some embodiments, to transmit a warning to an operator to stop harvesting crops that are too hot and/or to automatically stop harvesting.

Some embodiments provide for a system for use in connection with assessing characteristics of harvested specialty crops. In some embodiments, the system may be programmed to: (1) obtaining an image of a set of harvested specialty crops and associated depth information; (2) generating a 3D surface model (e.g., a 3D mesh or grid of cuboids) of the set of harvested specialty crops using the image and the depth information; and (3) estimating the volume of the set of harvested specialty crops using the 3D surface model. The estimated volume may be used, in turn, to determine the mass of the harvested specialty crops. This may be done in any suitable way and, for example, may be done by using the estimated volume and an estimated density for the particular type of specialty crops.

In some embodiments, the system estimates the volume of the set of harvested specialty crops by computing a volume integral using the 3D surface model. Computing the volume beneath the surface model may indicate the bulk volume of the harvested specialty crops.

In some embodiments, the 3D surface model comprises a plurality of sections, and the system computes the volume integral by estimating, using dimensions and depth information for each section of the 3D surface model, a sectional volume of harvested specialty crops in each section, to obtain a plurality of sectional volumes; and computing a sum of the plurality of sectional volumes. For example, the processor may compute the volume of each cuboid in a grid or the volume underneath each section of a 3D mesh.

In some embodiments, the techniques further include: accessing, in a database, a density for a type of specialty crop in the set of harvested specialty crops; and estimating a mass of the set of harvested specialty crops using the volume of the set of the harvested specialty crops and the density. This allows for the mass of all harvested specialty crops to be estimated without modifying the conveyor to include a scale (as some conventional specialty crop monitoring systems require). It should be appreciated that, in addition or instead of density, other types of information may be used together with geometric information about the harvested crops to estimate their mass. For example, characteristic shapes of each particular crop variety may be used to refine the volume and/or mass estimate of the harvested specialty crops. For instance, characteristic shapes of a particular type of harvested crop may be used together with lengths of major/minor axes of each individual crop of that variety to more accurately estimate the volume of each individual crop and, as a consequence, provide an improved estimate of its mass.

In some embodiments, the techniques further include: (1) obtaining a second image of a second set of harvested specialty crops and associated second depth information; (2) generating a second 3D surface model of the second set of harvested specialty crops using the second image and the second depth information; (3) estimating the volume of the second set of harvested specialty crops using the second 3D surface model; and (4) adding the estimated volume of the second set of harvested specialty crops to the estimated volume of the set of harvested specialty crops. In this way, multiple images of harvested specialty crops may be received and a running total of the volume of harvested specialty crops may be calculated. In some embodiments, the techniques may involve determining that there are specific specialty crops in both the first and second images and using features of the image to begin the second 3D surface model at an edge of the first 3D surface.

In some embodiments, the techniques further include: receiving location data indicative of a location at which the harvested specialty crops were harvested; and generating a map that associates the location with any information derived from the image of a set of harvested specialty crops and the associated depth information. Such information may be used for any suitable purpose. For example, the information may be used to generate a map of the proportions of USDA grade 1 and grade 2 potatoes that come from each portion of a field.

Some embodiments provide for a system for use in connection with assessing size, shape, variety, and/or quality of individual harvested specialty crops. Providing such information on a per-crop basis is a major advancement relative to conventional techniques that, at best, provide aggregate statistics of yield. Accordingly, in some embodiments, the system is programmed to perform: (1) obtaining an image of a set of harvested specialty crops and associated depth information; and (2) determining, using the image and the depth information, the size and/or shape of each of multiple specialty crops in the set of harvested specialty crops. Individualized size and/or shape information allows not only for the determination of quality for individual harvested specialty crops, but also provides a way to get refined volume and/or mass estimates of harvested specialty crops. In addition to or instead of determining size and/or shape of each of multiple specialty crops, in some embodiments, the system may be programmed to automatically determine variety of individual crops using the image and the depth information.

In some embodiments, determining the size and/or shape of each of multiple specialty crops in the set of harvested specialty crops includes: (1) applying an image edge detection technique to the image to obtain detected edges; (2) identifying, using the detected edges, boundaries of a first harvested specialty crop in the set of harvested specialty crops; and (3) determining, using the identified boundaries, a length of a major axis of the first harvested specialty crop and a length of a minor axis of the first harvested specialty crop. In some embodiments, a diameter of the specialty crop (e.g., diameter of a grape) may be determined in addition to or instead of the lengths of the major and/or minor axes. For example, by applying an edge detection filter to the image and clustering the detected edges, cross-sectional outlines of each harvested specialty crop can be obtained and measured. Identifying individual boundaries and/or dimensions enable further analysis of data regarding individual crops, for example, by analyzing the bounded portion of the image or a corresponding area of the depth information.

In some embodiments, the system is further programmed to compute using the length of the major axis distance and the length of the minor axis, a volume and/or a surface area of the first harvested specialty crop. For example, geometric assumptions about the harvested specialty crops (e.g., that potatoes are rotated ellipsoids) allow for individual volumes to be calculated from the major and minor axes.

In some embodiments, the system is further programmed to generate a 3D surface model of the set of harvested specialty crops using the image and the depth information; and estimate the volume of the set of harvested specialty crops using the 3D surface model. This allows for the volume of all harvested specialty crops to be computed. Individual volume measurements may be used to refine the estimated volume. The estimated volume may also be used to estimate the number of harvested specialty crops with a given level of quality.

In some embodiments, the system is further programmed to: (1) access a trained statistical model configured to output information indicative of harvested specialty crop quality (e.g., a USDA grade number or an indication of rot); (2) provide, as input to the trained statistical model, at least one feature selected from the group consisting of the image, the depth information, the length of the major axis, and the length of the minor axis; and (3) determine quality of crops in the set of harvested specialty crops based on output of the trained statistical model. For example, in some embodiments, an image depicting one or more individual harvested specialty crops may be provided as input to a neural network that is trained to detect rot (and/or any other factor indicative of quality examples of which are provided herein) on the surface of each of the one or more individual harvested specialty crops.

In some embodiments, additional information may be brought to bear on estimates of crop quality. For example, in some embodiments, weather and/or insect migration models may make the analysis of crop quality more precise (e.g., by allowing the techniques described herein to be more sensitive to certain weather and/or insect related crop disease).

It should be appreciated that the techniques introduced above and discussed in greater detail below may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

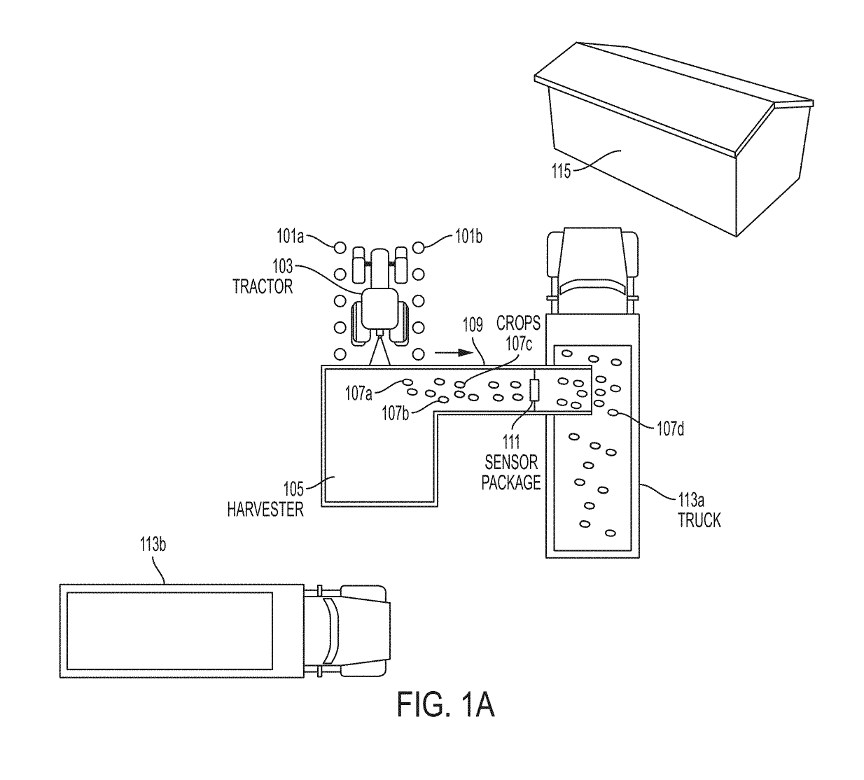

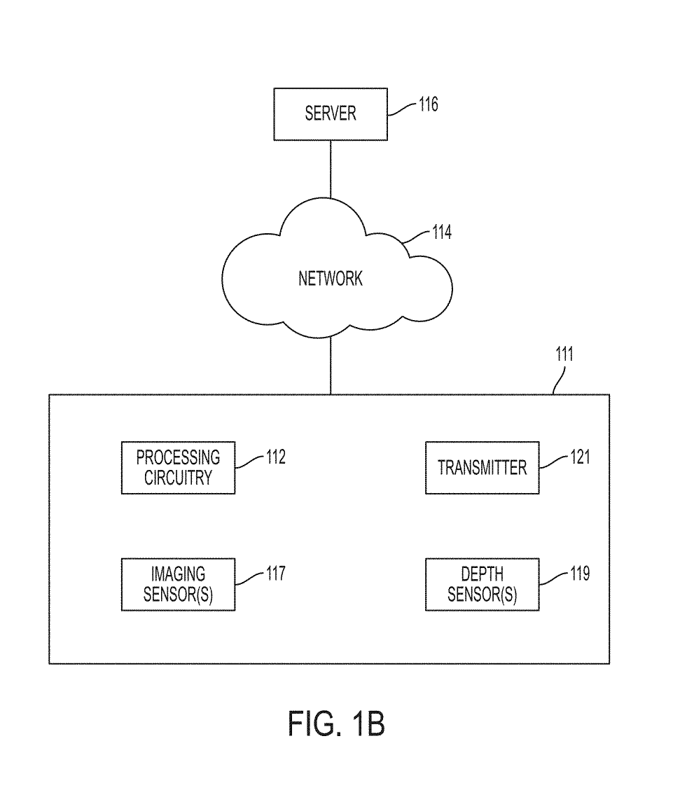

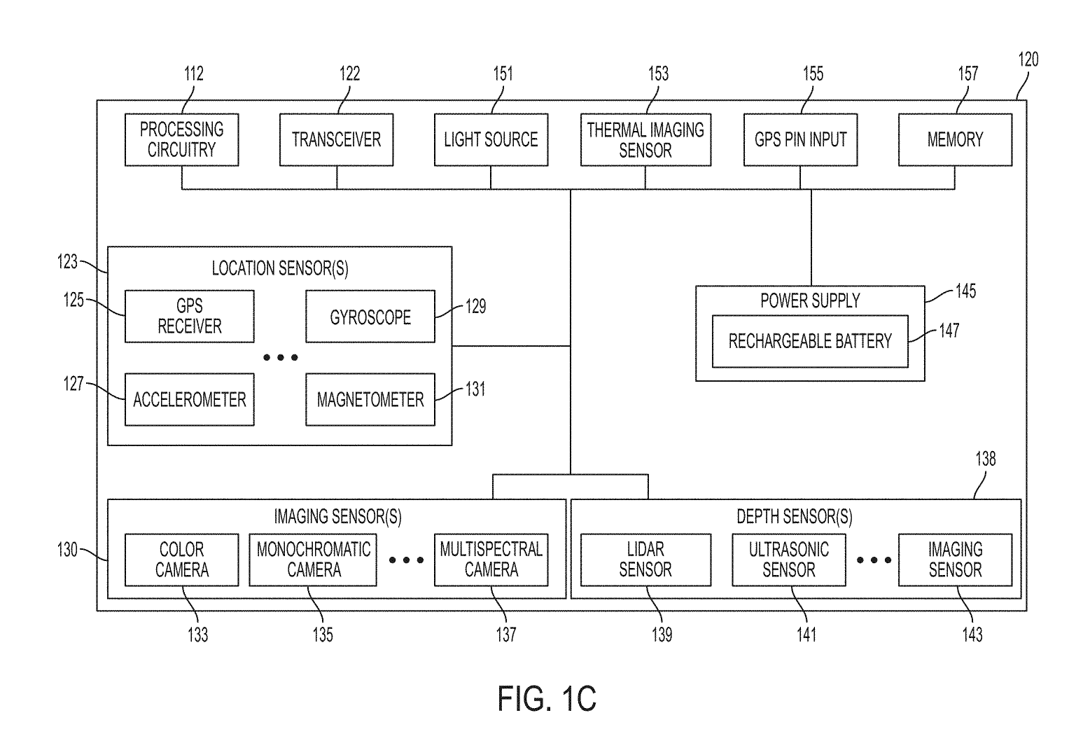

FIG. 1A shows an illustrative environment 100 in which some embodiments of the technology described herein may operate. FIG. 1A illustrates rows of plants 101a and 101b, a tractor 103, a harvester 105, harvested specialty crops 107a-d, conveyor 109, sensor package 111, trucks 113a and 113b, and storage facility 115. In the illustrative environment 100, rows of plants 101a and 101b are cultivated and bear specialty crops. The tractor 103 pulls the harvester 105 in order to harvest the specialty crops, which are deposited onto trucks 113a-b upon being harvested. The trucks transport the harvested specialty crops to storage facility 115. Prior to being deposited onto trucks 113a-b, at least some the harvested specialty crops may pass in the field(s) of view of one or more sensors in sensor package 111, which sensor(s) may collect data about the harvested specialty crops. In some embodiments, the collected data may be used to assess one or more characteristics of the harvested specialty crops (e.g., using imaging and depth data to determine crop weight, crop volume, crop yield, crop quality, etc.). It should be appreciated that although a handful of specialty crops are shown in FIG. 1A, this is only for clarity of illustration, as during operation the flow of crops is much more dense often and may be several layers thick.

In some embodiments, the tractor 103 may pull the harvester 105 in order to harvest the specialty crops. The tractor 103 may be operated by a human operator situated within the tractor or may be remotely operated. The tractor 103 may include a GPS receiver and/or other location sensor configured to obtain information specifying the position of the tractor. In some embodiments, the tractor 103 may include an onboard display that may be used to convey information to the human operator. Such a display may be used to provide to the human operator information about the harvested specialty crops including any information obtained by analyzing measurements made by one or more sensors in sensor package 111. For example, in some embodiments, the sensor package 111 captures successive images, compares features in the images, and determines the speed of the conveyor 109. In some embodiments, the sensor package includes a thermal imaging sensor that measures the temperature of the crops and alerts the operator if the crops are unsuitably hot for harvesting. In some embodiments, the sensor package 111 detects excessive moisture that may lead to having specialty crops rotting in storage. In some embodiments, analysis of images captured by the sensor package 111 may determine that specialty crops are being damaged (e.g., by detecting whether outlines of harvested crops deviate substantial from an expected geometry) and alert the operator to change harvesting settings. For example, a depth to which the harvester digs for crops may be increased (e.g., in response to detecting cuts on the harvested crops, which may appear bright white in imagery). In some embodiments, analysis of images captured by the sensor package 111 may determine that excessive soil is present on the harvester and the operator may be alerted to a need to decrease the depth to which the harvester 105 is digging. In some embodiments, the harvester 105 may be self-propelling such that a tractor is not required.

In some embodiments, the harvester 105 is configured to harvest specialty crops 107a . . . 107d and moves the harvested specialty crops 107a . . . 107d using conveyor 109 to one or more trucks (e.g., trucks 113a-b). Conveyor 109 may be a conveyor belt or any suitable mechanism for moving crops through the harvester. The harvester may be configured to harvest one or more particular types of specialty crops (e.g., a potato harvester, a tomato harvester, an onion harvester, etc.) and may be of any suitable type, as aspects of the technology described herein are not limited in this respect.

In some embodiments, the harvester 105 may include a GPS receiver and/or other location sensor configured to obtain information specifying the position of the harvester. In some embodiments, the harvester 105 may include a display for conveying information to a human operator. Such a display may be used to provide to the human operator information about the harvested specialty crops including any information obtained by analyzing measurements made by one or more sensors in sensor package 111. For example, the display in the harvester 105 may display any of the information discussed with reference to a display for the tractor 103 above.

In some embodiments, sensor package 111 may be coupled (e.g., mounted) to the harvester 105 in a position to collect data about specialty crops being harvested by harvester 105. The sensor package 111 may be contained in any suitable enclosure to house sensors and protect them from the operating environment. The sensor package 111 may include one or more sensors of any suitable type. In some embodiments, the sensor may include one or more imaging sensors (e.g., an RGB camera, a monochrome camera, a multi-spectral camera, etc.) and one or more depth sensors (e.g., one or more ultrasound sensors, one or more LIDAR sensors, one or more additional imaging sensors in addition to the one or more imaging sensors, etc.) for obtaining data about the specialty crops harvested by the harvester. In some embodiments, the sensor package 111 may be configured to capture one or more images of harvested specialty crops using the imaging sensor(s) and obtain depth data for the harvested specialty crops using the depth sensor(s). The image(s) and/or depth data may be transmitted to one or more external devices (e.g., one or more remote servers) for subsequent processing (e.g., to identify one or more characteristics of the specialty crops being harvested). Other examples of sensors that may be included in sensor package 111, in addition to or instead of the above-described sensors, are described herein including with reference to FIGS. 1B and 1C.

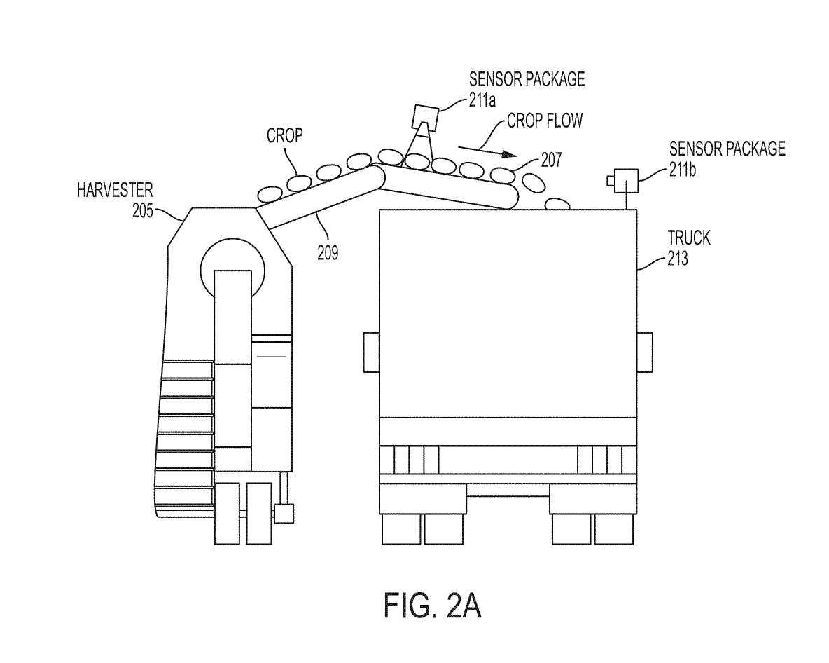

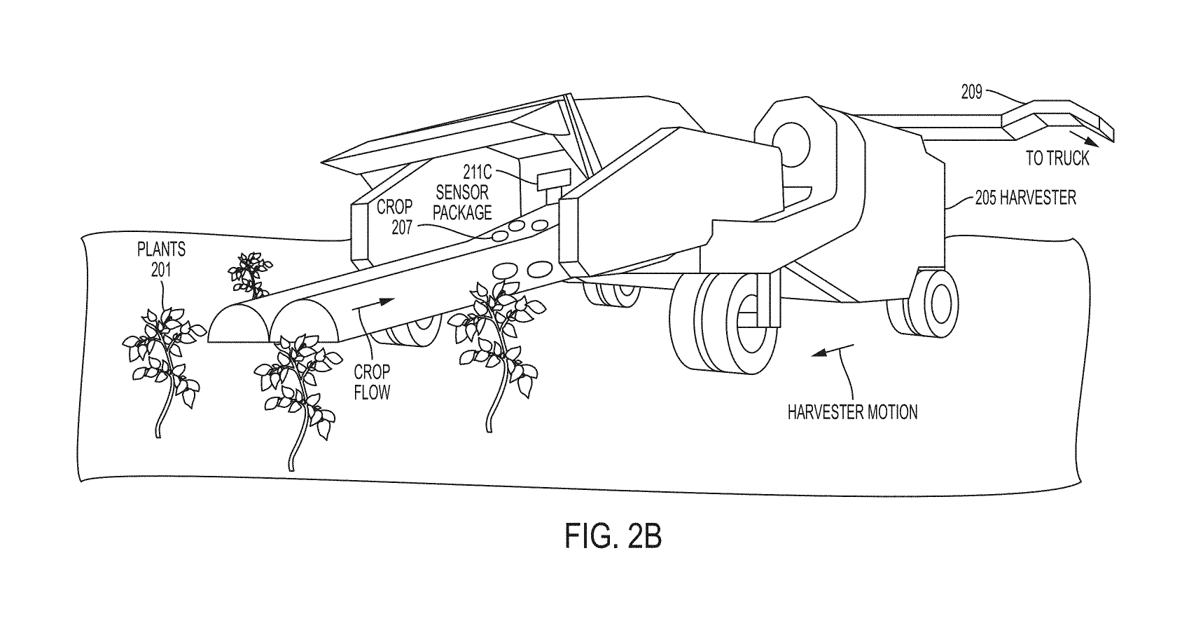

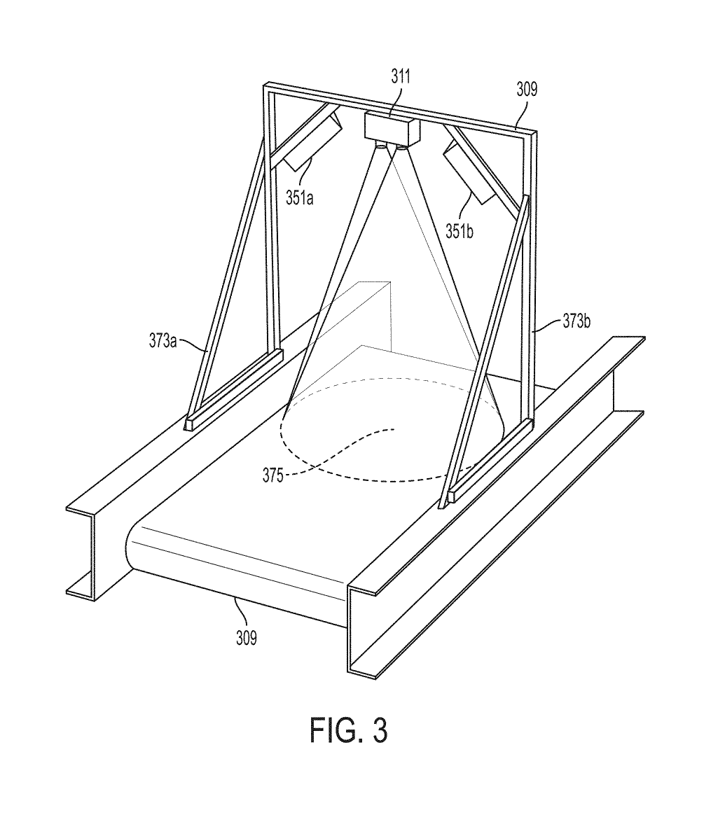

In some embodiments, the sensor package 111 may be positioned to collect data about at least some of the specialty being harvested by harvester 105. For example, in some embodiments, the sensor package 111 may be positioned above conveyor 109 with space for any harvested specialty crops 107 to pass between the surface of the conveyor 109 and the sensor package 111. An example of this is described herein including with reference to FIG. 2A and FIG. 3. As another example, in some embodiments, the sensor package 111 may be positioned proximate the intake of harvester 105. An example of this is described herein including with reference to FIG. 2B. As yet another example, in some embodiments, the sensor package 111 may be positioned to monitor crops from a side (sideways) rather than from above. Such placement provides a way of measuring depth of the crops along a different axis. It should be appreciated, however, that the sensor package may be coupled to a harvester in any other suitable way, as aspects of the technology described herein are not limited in this respect. For example, in some embodiments, the sensor package 111 is coupled to a conveyor belt or bin piler that is not coupled to a harvester. In addition, in some embodiments multiple sensor packages may be coupled to a single harvester. In such embodiments, the multiple sensor packages may be of a same type or may be of a different type (e.g., different sensor packages may include the same or different sensor(s)).

In some embodiments, the sensor package 111 may be used for continuous monitoring of specialty crops being harvested using harvester 105. The sensor package 111 may be used to obtain data about groups of harvested crops moving through the harvester. For example, the sensor package 111 may be used to obtain measurements of a set of harvested crops 107 (e.g., using one or more imaging sensors, one or more depth sensors, one or more thermal sensors, etc.) and, after the set of harvested crops 107 moves out of the field of view of one or more sensors in the sensor package 111 and another set of harvested crops moves into the field of view of the one or more sensors in the sensor package, the sensor package 111 may be used to obtain data about the other set of harvested crops. In this way, sensor package 111 may be used to gather information about specialty crops as they are being harvested by harvester 105. As described herein, data collected by sensor package 111 may be analyzed (e.g., by one or more computing devices physically remote from the sensor package 111) to determine one or more characteristics of the monitored crops (e.g., crop weight, crop volume, crop yield, crop quality, etc.).

FIG. 2A shows illustrative arrangements of one or more sensor packages in a field environment in which some embodiments of the technology described herein may operate. FIG. 2A illustrates a harvester 205, a conveyer 209, harvested specialty crops 207, sensor packages 211a and 211b, and truck 213. In the illustrated configuration sensor package 211a is coupled to harvester 205 and sensor package 211b is coupled to truck 213.

As shown in FIG. 2A, sensor package 211a is mounted to harvester 205 such that one or more sensors within sensor package 211a may obtain measurements of harvested specialty crops on the conveyor 209. Sensor package 211a may be mounted to harvester 205 by being mounted to conveyor 209 (e.g., as shown in FIG. 3). It should be appreciated, however, that sensor package 211 may be mounted to conveyor 209 in any other suitable way.

In some embodiments, the sensor package 211a may contain any of the numerous types of sensors described herein including, by way of example and not limitation, one or more imaging sensors (e.g., a color camera, a monochrome camera, or a multi-spectral camera, etc.) and one or more depth sensors (e.g., one or more ultrasound sensors, one or more LIDAR sensors, or one or more additional imaging sensors, etc.). In some embodiments, sensor package 211a may be configured to obtain information used for determining the distance between the harvested specialty crops on the conveyor 209 and the sensor package 211a. In configurations where the distance between the sensor package 211a and conveyor 209 may be fixed and known in advance (e.g., this distance may be determined during mounting of the sensor package 211a to conveyor 209), that distance together with measured distances between the sensor package 211a and one or more points on an individual crop may be used to determine the height of those points relative to the conveyor 209. In turn, the obtained height(s) may be used to estimate one or more characteristics of the individual crop (e.g., volume, weight, etc.).

As shown in FIG. 2A, sensor package 211b is mounted to the truck 213 such that one or more sensors within sensor package 211b may obtain measurements of harvested specialty crops as they transition from conveyor 209 to truck 213. In some embodiments, an imaging sensor or sensors within sensor package 211b may be used to image the flow of the harvested specialty crops 207 in order to measure the size and/or quality of individual crops. In some embodiments, sensor package 211b may be configured to measure the height, relative to the bed of truck 213, of one or more points on top of one or more harvested specialty crops 207 that have been deposited in the truck bed.

Although FIG. 2A illustrates two sensor packages deployed in a field environment for monitoring flow of harvested specialty crops, it should be appreciated that any suitable number of sensor packages may be used in some embodiments, as aspects of the technology described herein are not limited in this respect. For example, one, two, three, four, or five sensor packages may be deployed in the field environment in some embodiments. One or more sensor packages may be coupled to a harvester and/or one or more sensor packages may be coupled to each truck receiving harvested specialty crops from the harvester.

In some embodiments, where a sensor package is coupled to a harvester 205, the sensor package may be coupled to the harvester in any suitable way. For example, as shown in FIG. 2A, the sensor package may be mounted (e.g., above the conveyor 209) such that conveyor 209 is in its field of view. As another example, as shown in FIG. 2B with respect to sensor package 211c, the sensor package may be coupled to the harvester proximate to a crop intake of the harvester. Locating a sensor package proximate the crop intake may be advantageous in that it allows for the crops 207 to be accurately observed and associated with the exact location of harvesting regardless of if there is any pause in the conveyor 209, which may result from a truck filling with harvested specialty crops 207 and pulling away to storage.

FIG. 3 shows sensor package 311 in an illustrative configuration for monitoring harvested specialty crops on a conveyor belt, in accordance with some embodiments of the technology described herein. As shown in FIG. 3, sensor package 311 is mounted above conveyor 309 using mount 371, which is supported by uprights 373a and 373b at a fixed known height above the conveyor 309. In some embodiments, mount 371 may include one or more devices for dampening vibration of sensor package 311 (e.g., one or more shock absorbers). In some embodiments, sensor package 311 may be at least partially (e.g., fully) enclosed in an enclosure to minimize exposure of sensor package 311 to the environment (e.g., to dust, debris, dirt, etc.) and/or to control lighting conditions.

In some embodiments, the height may be factored into calculations of the depth of harvested specialty crops since some depth sensors measure to the top surface of harvested specialty crops, the depth of the specialty crops can be obtained through simple subtraction. The sensors within sensor package 311 may obtain data about any harvested specialty crops in field of view 375. The environment of sensor package 311 may be illuminated by one or more light sources (e.g., light sources 351a and 351b), which facilitates operation during low-light or night-time environments. For example, the light sources 351a and 351b are mounted on mount 371 so as to illuminate the field of view 375. In some embodiments, the uprights 373a and 373b may be adjustable and include sensors to communicate adjustments to the sensor package 311. For example, the height at which communication equipment (e.g., 4G antennas, GPS receiver, etc.) is mounted may be changed (e.g., made higher) by adjusting uprights 373a and 373b to improve signal reception.

Returning to FIG. 1A, after the harvested specialty crops are deposited into truck 113a which transports them to the storage facility 115, the truck 113b may replace the truck 113a at the output of the harvester 105 and receive the next batch of harvested specialty crops. In some embodiments, the sensor package 111 may compensate for pauses in the motion of conveyor 109 (e.g., for allowing a transition between the trucks 113a and 113b). For example, in some embodiments, to compensate for pauses in the motion of the conveyor 109, the sensor package 111 measures the amount of time for which the conveyor 109 was paused and the speed of the harvester 105 to estimate a distance traveled during the pause. In some embodiments, the sensor package stores location data indicating a path traveled by the harvester 105 during the pause and estimates a distribution for the backlog of crops along the path. In some embodiments, information about pauses in the conveyor belt may be used to adjust the 3D surface model of the flowing harvested specialty crops. For example, such a model may be shifted in time based on the length of a pause of the conveyor belt to compensate for the pause.

In some embodiments, the storage facility 115 may be any facility, for example a warehouse, shed, or silo, that is suitable for storing harvested specialty crops. In some embodiments, the yield of specialty crops and storage facility may be sufficiently large that a mapping of the quality of the harvested specialty crops to a location in the storage facility 115 may be useful for the commercialization or other utilization of the harvested specialty crops. For example, some buyers of harvested specialty crops require a certain minimum or average level of quality, and a priori knowledge of the quality of the specialty crops in the storage facility 115 can allow for efficient planning and utilization of the harvested specialty crops 107 to meet the required quality standards.

Accordingly, in some embodiments, information about crop yield and/or quality for a set of harvested specialty crops may be correlated with location information indicating where the set of harvested specialty crops is stored in storage facility 115. This may be done in any suitable way. For example, in some embodiments, information about crop yield and/or quality of a set of harvested specialty crops (e.g., derived from data obtained by a sensor package coupled to a specialty crop harvester, such as sensor package 111) may be associated with information identifying a truck (e.g., truck 113a) that transports the set of crops to a storage facility, and further with information indicating where in the storage facility the truck deposited the set of crops. As another example, in some embodiments, information about crop yield and/or quality of a set of harvested crops may be obtained from data gathered by a sensor package coupled to a bin piler in the storage facility, and this information may be associated with the position of the bin piler in the storage facility (which may be obtained by using a location sensor on the sensor package). In some embodiments, the position of the bin piler (e.g., in a local grid or polar coordinate system) within storage may be associated with any of the information (e.g., depth data, imaging data, and truck identification data) collected by the sensor package 111, and any specialty crop characteristics calculated therefrom, in order to generate a storage map.

It should also be appreciated that although in the illustrative embodiment of FIG. 1A, the crops being harvested are harvested from the ground using mechanical harvesting equipment, the techniques described herein may be applied to other harvesting situations. For example, the techniques described herein, may be applied to tree fruits and/or any other hand-harvested crops. For example, tree fruits may be picked and sent down a soft chute into a bin or onto a conveyor in the field, and a sensor package (e.g., sensor package 11) may be used to monitor the tree fruits in the bins and/or conveyor. As a specific non-limiting example, the techniques described herein may be applied to monitoring and assessing characteristics of hand-picked apples after they are placed in apple bins. After the apple bins arrive at a processing facility, the bins may pass under a sensor package (e.g., sensor package 111), which may generate an automated estimate of the volume and/or mass of harvested apples in each bin, the quality of one or more individual apples in each bin, an aggregate measure of quality of apples in each bin, an estimate of the variety of apples in each bin, etc. In some embodiments, a sensor package may be placed on the side of vehicle (e.g., a truck) to drive along fruit trees (e.g., apple trees in an orchard) to make estimates of yield for planning and/or marketing purposes.

FIG. 1B shows a block diagram of an illustrative sensor package 111 comprising a plurality of sensors for use in connection with monitoring and/or assessing characteristics of harvested specialty crops, in accordance with some embodiments of the technology described herein. As shown in FIG. 1B, sensor package 111 is configured to obtain one or more measurements using imaging sensor(s) 117 and/or depth sensor(s) 119, process at least some of the obtained measurements using processing circuitry 112 to obtain derived information, and transmit, via communication network 114, the measurements and/or information derived from the measurements using transmitter 121 to one or more remote computing devices such as, for example, remote server 116.

In some embodiments, the imaging sensor(s) 117 may include one or multiple imaging sensors. An imaging sensor may be configured to capture one or more images of harvested specialty crops. In some embodiments, an imaging sensor may be a camera. For example, an imaging sensor may be a color camera, a monochrome camera, a multi-spectral camera, and/or a device configurable to operate as one or more of a color camera, a monochrome camera, and a multispectral camera. As another example, an imaging sensor may include a charge-coupled device (CCD) imaging sensor, a complementary metal oxide semiconductor (CMOS) imaging sensor, an n-channel MOSFET (NMOS) imaging sensor, and/or any other suitable imaging sensor, as aspects of the technology described herein are not limited in this respect.

In some embodiments, the imaging sensor(s) 117 may be ruggedized and/or weather proof. For example, the imaging sensor(s) 117 may be partially enclosed and/or sealed off from the operating environment. In some embodiments, the imaging sensor(s) may be entirely enclosed and/or sealed off from the operating environment. In such configurations, the enclosure may include a window (e.g., a transparent window) through which the imaging sensors may image the harvested specialty crops.

In some embodiments, the imaging sensor(s) 117 may be configured to capture a series of images of harvested specialty crops. The images in the series may be captured at set time intervals or in a video stream.

In some embodiments, the image(s) captured by the imaging sensor(s) 117 may be used for determining one or more characteristics of harvested specialty crops. For example, the obtained images may be used to measure geometric characteristics of the harvested specialty crops. In some embodiments, for example, an image may be analyzed by detecting points on the edges of the individual crops (e.g., using any suitable edge detection technique), clustering the points to generate sets of points associated with individual crops, and using the generated sets of points to detect lengths of the major and minor axes for each individual crop. In turn, the lengths of the major and/or minor axes may be used to estimate the volume of the individual crop.

As another example, in some embodiments, the image(s) captured by the imaging sensor(s) 117 may be used to determine the quality of one or more individual harvested specialty crops. For example, in some embodiments, at least a portion of an image (e.g., the entire image) may be provided as input to a trained statistical classifier, which may provide output indicating for each of one or more harvested specialty crops in the image, a measure of quality of the crops (e.g., a measure indicating whether one or more of the crops have abrasions, bruising, scratch, graze, rupture, bleaching, greening, rot, decay, rough spots, sprouting, lesions, wilting, and/or any other suitable quality characteristics). In some embodiments, such statistical models may be trained through the use of labeled training data. The labeled training data may be obtained at least in part through crowd-sourcing (whereby people use a web-based crowd-sourcing platform to label individual crops in images using different types of quality labels), in some embodiments. In this way, a large number ordinary people not necessarily having any agricultural training (rather than solely farmers, agronomists, researchers, etc.) may label images to obtain labeled training data which may be used to train one more statistical models.

As another example, in some embodiments, the image(s) captured by the imaging sensor(s) 117 may be used to determine the variety of one or more individual harvested specialty crops. For example, the image(s) captured may be used to determine the variety of harvested potatoes, the variety of harvested apples, the variety of harvested tomatoes, etc. In some embodiments, geometric characteristics of the crops may be automatically derived from the images (e.g., using the techniques described herein) and the variety may be determined automatically from the geometric characteristics. In other embodiments, a machine learning approach may be used. For example, an image of harvested specialty crops may be provided as input to a trained statistical model (examples of which are provided herein) and output of the trained statistical model may provide an indication of the variety of the crop(s) in the image.

As another example, in some embodiments, the image(s) captured by the imaging sensor(s) 117 may be used for determining the depth of one or more individual harvested specialty crops. For example, the image(s) may be used as part of a stereoscopic vision process whereby images concurrently captured by two imaging sensors are used to determine the depth of objects (e.g., crops) in an image. For example, in some embodiments, a correspondence between features in pairs of images may be computed to generate a disparity map, which together with information about the relative position of the two cameras to each other may be used to determine depths of one or more points in the image. As another example, the image(s) may be used as part of a monoscopic vision process, whereby images captured by the same imaging sensor at two different points in time (e.g., due to motion of the conveyor belt) may be used to determine depths of one or more objects in the image. Any suitable stereoscopic or monoscopic vision process may be used, as aspects of the technology described herein are not limited in this respect.

As another example, in some embodiments, the image(s) captured by the imaging sensor(s) 117 may be used to discriminate between harvested specialty crops and debris. In some embodiments, a color camera can be used to discriminate between a specialty crops, tare, soil, and rocks by comparing the colors of areas of the image(s). For example, imagery of potatoes may be used to estimate tare. A percentage of “coverage” of dirty on an individual potato, coupled with the size of the individual potato, may provide an estimate of tare. For instance, since potatoes can be light colored (e.g., yellow), tare may be estimated by identifying and counting the number of dark cells (e.g., pixels) in an image. Using a thermal imaging sensor allows this analysis to be refined by taking into account differences in emissivity since the presence of tare on an individual potato will change its thermal emissivity. Thus, for example, tare may be estimated by identifying the number of cells (e.g., pixels) that are dark (e.g., an amount of light detection is below a threshold) and have different emissivity that a threshold emissivity.

In some embodiments, the imaging sensor(s) 117 are configured to capture one or more electromagnetic spectra. For example, the imaging sensor(s) 117 may include one or more near infrared sensors configured to detect emissions in the 700 nm-2500 nm range. In some embodiments, the spectral absorption and reflection from the harvested specialty crops can be compared to a database of spectral data in order to determine a variety of specialty crop. In some embodiments, debris exhibits different spectral characteristics from the harvested specialty crops. The spectral difference may be determined by, for example, computing an average spectral response for the specialty crops or accessing a typical spectral response in a database and comparing a portion of the multispectral image using subtraction, dynamic frequency warping, or any suitable method.

As another example, in some embodiments, the image(s) captured by the imaging sensor(s) 117 may be used to determine the speed of conveyor moving the passing harvested specialty crops. In turn, this speed may be used to calculate the volume and yield of harvested specialty crops and/or to guide the harvesting operation. In some embodiments, the processing circuitry 112 and/or the server 116, matches features present in two images, of harvested specialty crops, captured successively after a measured or predetermined interval of time, in order to determine the distance traveled by the common features and, therefore, the average speed of the harvested crops and conveyor during the time interval. In some embodiments, the estimated speed may be communicated to an operator of a harvester and/or bin piler. In some embodiments, the estimated speed may be used to adjust the speed of the conveyor automatically, without user input. For example, when crops are piling up too fast, the speed of the conveyor may be reduced.

In some embodiments, the depth sensor(s) 119 may include one or multiple depth sensors. A depth sensor may be configured to measure information related to the depth of one or more of the harvested specialty crops.

In some embodiments, the depth sensor(s) 119 may include one or more ultrasonic sensors, which transmits ultrasonic waves and uses the timing of the reflection to determine the distance between the harvested specialty crops and the depth sensor(s) 119. Multiple ultrasound sensors may be arranged in a one- or two-dimensional array. In some embodiments, depth measurements made by the ultrasound array may be used to generate a point cloud of the depths of crops flowing over the conveyor belt. In turn, this point cloud may be used to generate a 3D mesh that can be used to estimate the volume of the crops flowing over the conveyor belt. At least some of the distance measurement made by the ultrasound array may be geotagged, which allows the volumetric flowrate of crops over the conveyor belt to be associated with the location in a field from which the specialty crops were harvested.

In some embodiments, the depth sensor(s) 119 may include one or more LIDAR sensors configured to create a two- or three-dimensional point cloud of measurements of the distances between the sensor and the harvested specialty crops. The LIDAR sensor may be configured to continuously scan a flow of harvested specialty crops. In some embodiments, the LIDAR sensor may be configured to compute a 3D line scan of the 3D crops. Inclusion of one or more LIDAR sensors provides high resolution depth information. In some embodiments, the point cloud generated by the LIDAR sensor(s) may be used to generate a 3D mesh that can be used to estimate the volume of the crops flowing over the conveyor belt. At least some of the distance measurements made by the ultrasound array may be geotagged, which allows the volumetric flow rate of crops over the conveyor belt to be associated with the location in a field from which the specialty crops were harvested.

In some embodiments, the depth sensor(s) 119 may include one or more imaging sensors. For example, a depth sensor may be a color camera or a monochrome camera, and/or a an imaging sensor configurable to operate as one or more of a color camera, a monochrome camera, and a multispectral camera including an a charge-coupled device (CCD) imaging sensor, a complementary metal oxide semiconductor (CMOS) imaging sensor, an n-channel MOSFET (NMOS) imaging sensor, and/or any other suitable imaging sensor, as aspects of the technology described herein are not limited in this respect. In such embodiments, data obtained by the depth sensor 119 may be used together with data obtained by one more imaging sensor(s) 117, for example by using a stereoscopic vision process, to determine information indicating the depth of one or more specialty harvested crops. The depth information may be processed similarly to what was described for the ultrasound and LIDAR point clouds. For example, the depth information may be used to generate 3D surface model from which the volume of flow of crops may be determined.

Although in the illustrated embodiment, imaging sensor(s) 117 are shown as being physically different sensors from depth sensor(s) 119, in some embodiments, a single imaging sensor may be used as a depth sensor, with no additional sensors required. In some such embodiments, the imaging sensor may be used to capture a series of images as crops are moved by a conveyor built. In turn, the captured images and information about the speed of the conveyor's motion to determine the depth of one or points in the image using any suitable monoscopic imaging technique.

As described herein, any of numerous types of sensors may be configured to collect information related to the depth of the harvested specialty crops (e.g., using LIDAR or ultrasound sensors) or data from which such information may be derived (e.g., using multiple images collected by one or more imaging sensors and stereoscopic or monoscopic vision techniques). In some embodiments, information related to the depth of the harvested specialty crops may comprise information indicating one or more distances measured from the depth sensor(s) 119 to surfaces of harvested specialty crops. In some embodiments, the distance between the depth sensor(s) 119 and the conveyor moving the harvested specialty crops or the bottom of a storage container holding harvested specialty crops may be known and used to calculate the depth of harvested specialty crops by subtracting the distance between the surface of the specialty crops and the depth sensor(s) 119. In some embodiments, the depth sensor(s) 119 is configured to measure the distance between the conveyor moving the harvested specialty crops or the bottom of a storage container holding harvested specialty crops, for example by obtaining depth information when/where the imaging sensor indicates that no harvested specialty crops are within range of the sensor package 111, by monitoring a minimum depth measurement that is assumed to represent a void in harvested specialty crops, or by analyzing the depth information (e.g., when a sensor reading includes a consistent measurement, echo, or reflection that is deeper than any harvested crop and determined to be the maximum depth).

In some embodiments, the processing circuitry 112 may convert depth information obtained from the depth sensor(s) 119 to a compressed format to reduce the amount of information transmitted by sensor package 111, which may be advantageous for rural areas (e.g., agricultural fields) with wireless data networks having limited bandwidth that cannot transmit high-resolution data sets obtained by the depth sensor(s) 119. Accordingly, in some embodiments, the processing circuitry 112 compresses depth information to obtain lower-resolution depth information and transmits the lower-resolution depth information instead of the originally-obtained depth information.

For example, in some embodiments, processing circuitry 112 may divides the depth information into a virtual grid having multiple sections (e.g., a 3×3 grid, a 5×5 grid, a 10×10 grid, a 5×10 grid, a 20×20 grid, or any other suitable size/resolution grid) and computes an average depth in each section of the grid, or any suitable metric for representing the depth information in each section of the grid. In this way, the original depth information may be transformed to compressed depth information specified with respect to a coarse resolution grid.

As another example, in some embodiments, the processing circuitry 112 generates a surface mesh from the depth information. Points in the surface mesh may be placed at regular intervals, as a function of the gradient of the depth, as a function of the absolute value of the depth, or by any suitable means. For example, in some embodiments, a surface mesh may be generated as a grid with the height of each grid element (e.g., squares, rectangles, etc.) being determined as a function (e.g., as an average, a median, etc.) of depths of points in the grid element. As another example, a surface mesh may be generated from the depth measurements, without averaging, using Delaunay triangulation. In some embodiments, the processing circuitry 112 computes a series of approximate isoclines, for example using a k-means clustering algorithm, a flood fill algorithm, or any suitable clustering, filtering, and/or rounding technique, that are used to represent areas of sufficiently equal depth, e.g., accurate enough to keep the volume measurement within a given tolerance. In this way, the original depth information may be transformed to a representation of the surface mesh, transmitting which may involve transmitting less information than would be required to transmit the uncompressed depth information.

In some embodiments, the processing circuitry 112 may be a microprocessor, programmable logic device, field programmable gate array (FPGA), application specific integrated circuit (ASIC), or any other suitable processing circuitry in electrical communication with the sensors and additional elements (e.g., the imaging sensor(s) 117, the depth sensor(s) 119, and the transmitter 121) of the sensor package 111 in order to provide and process inputs and outputs of the devices.

In some embodiments, the processing circuitry 112 may be configured to obtain depth information based on depth data from the depth sensor(s) 119. In some embodiments, the depth data may be multiple images and the processing circuitry may be configured to generate depth information from the images using a stereoscopic or monoscopic vision pipeline. In some embodiments, the depth data include a large volume of data and the processing circuitry 112 may be configured to compress the depth data to produce compressed depth data. This may be done in any suitable way including in any of the ways described above.

In some embodiments, the processing circuitry 112 may be configured to associate data gathered by one or more sensors of sensor package 111 with metadata containing information indicating the location (e.g., a location in the field, a location in a storage facility) of where the data was gathered. For example, the processing circuitry may associate an image obtained by an imaging sensor 117 with information indicating a location (e.g., coordinates, location on a map, etc.) in the field or storage facility at which the image was captured. Additionally or alternatively, the processing circuitry 112 may be configured to associate data gathered by one or more sensors of sensor package 111 with metadata containing information indicating a time at which the data was gathered. In this way, in some embodiments, at least some or all of the data collected by the sensor package 111 may be geospatially and/or temporally tagged.

In some embodiments, the processing circuitry 112 may be configured to associate data gathered by one or more sensors of sensor package 111 with metadata provided by a tractor. Non-limiting examples of metadata provided by a tractor (via ISOBUS port and/or in any other suitable way) include GPS data, pressure data, pressure, and speed.

In some embodiments, the processing circuitry 112 may receive an indication from harvesting equipment to which it is mounted (e.g., a harvester 105 or bin piler) that a conveyor (e.g., conveyor 109) is in motion, and in response enable one or more components of the sensor package 111. For example, the processing circuitry may enable at least one of the imaging sensor(s) 117, the depth sensor(s) 119, and the transmitter 121.

In some embodiments, the transmitter 121 is configured to transmit data gathered by one or more sensors in sensor package 111 and/or information derived therefrom to one or more remote computing devices. For example, transmitter 121 may be configured to transmit one or more images captured by the imaging sensor(s) 117. As another example, transmitter 121 may be configured to transmit depth information generated from data collected by the depth sensor(s) 119. In some embodiments, transmitter 121 may be a wireless radio (e.g., configured for Bluetooth, 802.11 Wi-Fi, Cellular, or Near Field Communication networking), a wired transmitter, a UART, a USB controller, and/or any suitable transmitter/transceiver.

In some embodiments, the data generated by sensor package 111 may be accessed through an application programming interface (e.g., API). In this way, the sensor data, which may be geotagged and/or temporally-tagged, may be incorporated into conventional farm management software (e.g., MyJohnDeere portal, FarmLogs, etc.).

In some embodiments, the transmitter 121 may transmit data about one or more harvested specialty crops over the network 114 (e.g., a local area network, a wide area network, the Internet, etc.) to server 116, which can store and/or further process the received data. In some embodiments, training data for training statistical models for crop quality and/or variety determination may be aggregated using network 114. In some embodiments, updates to sensor package 111 may be pushed via over-the-air updates using network 114. Server 116 may process received data to determine one or more characteristics of the harvested specialty crops including size, volume, quality, yield, and the like. Performing such processing on server 116, rather than sensor package 111, is advantageous because it reduces the memory and processing power requirements for the sensor package, thereby reducing its cost and complexity. However, it should be appreciated that, in some embodiments, sensor package 111 may be configured to process received data to determine one or more characteristics of the harvested specialty crops, as aspects of the technology described herein are not limited in this respect.

In some embodiments, the transmitter 121 may be configured to transmit information to a harvester (e.g., harvester 105) or tractor (e.g., tractor 103) or a bin piler, for example through a connection to an instrument or display panel, in order to convey information to the operator of the equipment. In some embodiments, the transmitter 121 may be configured to transmit information to a proximate cell phone or a nearby office, e.g., one occupied by a farm supervisor.

In some embodiments, the server 116 may be a single computing device or a collection of computing devices. For example, server 116 may be one or more servers, one or more processing nodes part of a cloud-computing service, and/or any other suitable collection of one or multiple (physical or virtual) devices, as aspects of the technology described herein are not limited in this respect.

In some embodiments, the server 116, using a connection to the network 114, may obtain an image captured by imaging sensor(s) 1117 of a set of harvested specialty crops and associated depth information measured by depth sensor(s) 119. In some embodiments, using the received image and associated depth information, the server 116 may generate a three-dimensional (3D) surface model of the set of harvested specialty crops. In some embodiments, the depth information may include a grid of depth measurements, in which case the 3D model may be considered to be a collection of cuboids with cross-sectional dimensions equal to the dimensions of each grid section, which may or may not be uniform, and heights equal to the depth measurement in each grid section. In some embodiments, the 3D model may be a surface mesh in which points representing depth information are connected with vectors. The surface mesh may be received by server 116, in some embodiments, or generated by server 116 in other embodiments. The points in the surface mesh may be placed at regular intervals (e.g., at intersections on a grid), at locations selected based on the depth information, or in any other suitable way, as aspects of the technology described herein are not limited in this respect.

In some embodiments, the server 116 may receive a series of images and associated depth information from the sensor package 111 and may iteratively operate on the information and update the 3D model. In some embodiments, the server 116 may determine features, such as patterns of pixel values, which are common to multiple images of the harvested set of specialty crops and use the common features to determine an offset distance representing the distance the harvested specialty crops moved in-between the capture of the images. The offset distance may be calculated using information received from the sensor package 111 and/or calculated by the server 116 to represent the speed of the specialty crop conveyor (e.g., conveyor 109). The server 116 may use the offset distance to avoid double counting any of the harvested specialty crops and may stitch together multiple images or supplement the 3D surface model with an adjacent 3D surface model of the new harvested specialty crops.

In some embodiments, the server 116 may estimate the volume of harvested specialty crops using the 3D surface model. For example, the server 116 may compute a volume integral of the area underneath the 3D surface model using any suitable mathematical software. As one example, when the 3D surface model includes a collection of portions (e.g., cuboids), the server 116 may calculate the volume integral by calculating the volume of the portions of the surface model and sum the calculated portion volumes. In some embodiments, the server 116 may sum portions that are perpendicular to the direction of the flow of crops on the conveyor and pass under the sensor package 111 at the same time in order to account for crops that were harvested in substantially the same location or at the same time for association with sensor data.

In some embodiments, the server 116 may access one or more databases to determine the density of a given specialty crops, which may be identified by a harvester operator, a human observer, image analysis, or in any other suitable way. The server 116 may use the density and the estimate volume to determine the mass of harvested specialty crops. In some embodiments, the server may also estimate, based on the received images (e.g., based on color information), a portion of the flow of material that is debris and deduct and estimated volume of debris from the 3D surface model volume measurements.

In some embodiments, the server 116 may be configured to process the received data to determine the size and/or shape of each of multiple individual harvested specialty crops. This may be done in any suitable way. For example, in some embodiments, the server 116 first uses the image from the imaging sensor(s) 117 to detect the edges of the harvested specialty crops by applying edge detection image processing techniques. Then server 116 applies a clustering algorithm (e.g., k-means clustering, hierarchical clustering, or any other suitable clustering algorithm) to the detected edges to determine which edges belong to an individual harvested specialty crop. From the clusters of edges, the server 116 may then determine geometric characteristics of each of one or more individual crops including, for example, a length of the major axis of each crop and/or a length of a minor axis of each crop.

In some embodiments, the major and minor axes may be found by searching the space of pairs of points on the edge of a specialty crop. For example, distances between all pairs of points on the edge of the specialty may be computed and the longest distance is determined to be the major axis. In some embodiments, the minor axis may be chosen to be the longest distance between edges that is perpendicular to the major axis. In some embodiments, the shortest distance from each point to any point on the opposite side of the major axis is computed and the minor axis is determined to be the longest such distance. In some embodiments the space of distances between points on the edge of the specialty crops is searched using any suitable means, e.g., by using a hill climbing algorithm.

In some embodiments, in order to determine the lengths of the major and/or minor axis, processing may be performed to determine a “length” represented by a pixel in an image. This length may be determined using depth information received by server 116. In some embodiments, the depth information is checked at a centroid of the individual specialty crop (e.g., where the major and minor axes intersect) and used to convert all pixel values to lengths for the harvested specialty crop. In some embodiments, the depth value at the centroid is also used as a third axis in modeling the harvested specialty crop as an ellipsoid.

In some embodiments, the lengths of the minor and major axes of an individual harvested specialty crop may be used to provide an ellipsoidal approximation to the volume of the crop according to:

(π/6)(minor axis length)²(major axis length).

In some embodiments, the server 116 may compute a volume integral over a length of the major axis and computing the area of each cross-sectional circle with the radius being the distance from the major axis to the edge in the image. These methods for computing individual volume are provided for example and not limiting, as the server 116 may use the image and/or depth information to calculate the volume of individual harvested specialty crops in any other suitable way. In some embodiments, the individual volumes of specialty crops may also be used to refine the bulk volume measurements of volume.

In some embodiments, the server 116 may be configured to determine the quality of individual specialty crops from the data received (e.g., one or more images and associated depth information). This may be done in any suitable way. In some embodiments, one or more geometric characteristics of an individual crop (e.g., volume, length, diameter, length or major axis, length of minor axis, etc.) may be used to estimate the quality of the crop. For example, the lengths of the major and minor axes may be used to determine whether an individual potato is graded as a “USDA No. 1” potato or a “USDA No. 2” potato.

In some embodiments, the server 116 may be configured to use one or more trained statistical models to analyze information received from the sensor package 111 in order to determine the quality of individual harvested specialty crops. For example, the server 116 may provide an image depicting at least one individual specialty crop as input to a trained statistical model and the output of the trained statistical model may provide an indication of the quality of the individual specialty crop. For example, the output of the trained statistical model may indicate whether the individual specialty crop has rot, bruising, discoloration and/or any other characteristic indicative of its quality. Examples of quality characteristics are provided herein. Non-limiting examples of trained statistical models include a neural network (e.g., deep neural network, convolutional neural network, etc.), a Bayesian classifier, a decision tree, a support vector machine, a Gaussian mixture model, a graphical model, a generative model, a discriminative model, a statistical model trained using supervised training, a statistical model trained using unsupervised training, and/or any other suitable type of trained statistical model.

In some embodiments, the server 116 may provide information related to the harvested specialty crops, including captured images, to human users who can detect rot or other measures of quality and communicate their results back to the server 116. In this way, human users may be used to label training images and the obtained training data, comprising images and associated labels, may be used to train (e.g., estimate parameters of) a statistical model to obtain a trained statistical model.

In some embodiments, the server 116 may be configured to output any of the calculated geometric or quality information to a memory, a user, or any suitable destination. In some embodiments, the server 116 may be configured to store the yield and quality information in a database for later recall and analysis by a user. In some embodiments, the server 116 may be configured to generate yield statistics and transmit the statistics to the grower of the specialty crops.

In some embodiments, the server 116 may be configured to provide feedback used to guide harvesting of the specialty crops, for example by transmitting information to the sensor package 111 or a user associated with the sensor package 111. In some embodiments, the image data may reveal specialty crops that are being damaged by the harvesting, that conditions, such as moisture or temperature, are suboptimal for harvesting, or that the harvested crops contain high levels of impurities. In some such embodiments, the information provided by server 116 may be used to alter operation of the harvesting equipment (e.g., by shutting down harvesting, slowing down the harvesting rate, changing one or more configurations of the harvester, etc.).

FIG. 1C shows a block diagram of another illustrative device comprising a plurality of sensors for use in connection with monitoring and/or assessing characteristics of harvested specialty crops, in accordance with some embodiments of the technology described herein. As shown in FIG. 1C, illustrative sensor package 120 is configured to obtain location data using location sensor 123, obtain imaging data and depth data that may be associated with the location data using imaging sensor(s) 130 and depth sensor(s) 138, and process at least some of the obtained measurements using processing circuitry 112, and transmit processed information using transceiver 122. Additionally, the sensor package 120 includes power supply 145 including rechargeable battery 147, light source 151, thermal imaging sensor 153, GPS pin input 155, and memory 157. The location sensor(s) 123 includes GPS receiver 125, accelerometer 127, gyroscope 129, and magnetometer 131. The imaging sensor(s) 130 includes color camera 133, monochromatic camera 135, and multispectral camera 137. The depth sensor(s) 138 includes LIDAR sensor 139, ultrasonic sensor 141, and imaging sensor 143.

Similarly to FIG. 1B, the sensor package 120 contains processing circuitry 112 in electrical communication with a plurality of sensors. Additionally, processing circuitry 112 is coupled to memory 157 that can be used to store sensor outputs and any associations there between (e.g., association between depth data and an image, association between location data and an image, association between LIDAR and ultrasonic data, or association between color and multispectral images).

The power supply 145 may be used to power the various sensors in the sensor package 120. In some embodiments, the power supply 145 may be configured to receive power from harvesting equipment that may be used to power sensor and charge the rechargeable battery 147. In some embodiments, power received from the harvester is an indication that harvesting of specialty crops has begun and the processing circuitry enables the sensors of the sensor package 120 in response.

In some embodiments, the power supply 145 includes the rechargeable battery 147. In some embodiments, the rechargeable battery is a lithium ion, lithium polymer, nickel-metal hydride, or any other suitable type of rechargeable battery. In some embodiments, the sensor package 120 includes one or more non-rechargeable batteries.

In some embodiments, the rechargeable battery 147 may be used as a backup power supply, or un-interruptible power supply, to provide power to properly shut down the sensors and processing circuitry 112 when power from the harvester, bin piler, or other harvesting-related equipment is lost.

The transceiver 122 may be any suitable transceiver(s) or a combination of separate transmitters and receivers and transmit any of the output from the sensors in the sensor package 120. In some embodiments, the transceiver is configured to receive near field communication (NFC) or RFID data from a truck (e.g., 113a) that is receiving the harvested specialty crops. The NFC or RFID data indicates an identification of the truck and can be used to map the yield of the harvested crops to a truck and to the location where the truck stores (e.g., in storage facility 115) a load of specialty crops.

The imaging sensor(s) 130 includes multiple imaging sensors configured to image harvested specialty crops in a variety or spectra and formats. Any of the imaging sensors included in the imaging sensors(s) 130 may be configured capture images at set time intervals, including intervals sufficiently short to be considered video capture. In some embodiments, two or more of the imaging sensors 130 may be configured to capture images concurrently. Images captured at different times using different imaging sensors 130 may be aligned based on features derived from the images, the speed of the conveyor, a measured difference in the times at which the images were captured, and/or any suitable combination thereof.

In some embodiments, one or more of the imaging sensors 130 may operate in the visible spectrum. In some such embodiments, the sensor package 120 includes the light source 151 to illuminate the harvested specialty crops to allow the imaging sensor(s) to operate consistently at night or other low-light conditions.

In some embodiments, the color camera 133 may be configured to capture color photos of the harvested specialty crops, e.g., using a standard RGB color format. Color image data may be used to discriminate between debris and specialty crops, for example tare, rocks, and potatoes may all typically be different shapes and colors.

In some embodiments, the monochromatic camera 135 may be used by sensor package 120 to capture monochromatic images of the harvested specialty crops. This may be advantageous because a monochromatic image requires less bandwidth to transmit for further processing, while still being suitable for assessing various characteristics of the harvested specialty crops.

In some embodiments, the multispectral camera 137 may be used by sensor package 120 to capture images of the harvested specialty crops in one or more different spectra outside the visible spectrum. The spectral signature of the harvested specialty crops may be used to identify the specialty crops being harvested, for example, by comparing spectral data to values retrieved from a database. In some embodiments, the multispectral camera 137 may capture a broad range of wavelengths of light that is processed using one or more band-pass filters.

In some embodiments, any captured images, which may be processed in real time or stored in the memory 157, may be associated with location data from the location sensor 123 to indicate where each individual image was taken, and transmitted using the transceiver 122 to any suitable receiver.

The sensor package 120 also includes the thermal imaging sensor 153 configured to capture thermal images of the harvested specialty crops. In some embodiments, the thermal imaging sensor 153 may be configured to capture data indicating the temperature of the harvested specialty crops. The temperature may be displayed to the operator of the harvester to indicate whether temperature conditions are suitable for harvesting. In some embodiments, the thermal imaging sensor 153 may include an infrared sensor. In some embodiments, operators at processing facilities may use information provided by the thermal imaging sensor (and/or one or more other sensors) to reject a load of crops, for example, because the temperature of the crops is too high and/or there is too much tare.

The depth sensor(s) 138 may include any of the types of sensors described with reference to FIG. 1B including, for example, one or more ultrasound sensors, one or more LIDAR sensors, and/or one or more imaging sensors.

Location sensor(s) 123 may include one or more sensors configured to measure the location of the sensor package 120. In the illustrative example of FIG. 1C, location sensor(s) 123 include the GPS receiver 125. In some embodiments, the GPS receiver 125 is configured to receive GPS location data from an external source, such as a tractor or harvester. The GPS receiver may rely on standard GPS signals, e.g., those with a resolution of 3-15 feet, or differential GPS signal, which may be accurate within sub-inch accuracy. In the illustrated embodiment, the location sensor(s) 123 includes an accelerometer 127, a gyroscope 129, and a magnetometer 131 collectively forming an inertial measurement unit (IMU), though in other embodiments, none, any one, or any of two of these sensors may be used. The inertial measurement units measure the relative motion of the sensor package 120, and can be used to create a local coordinate system. Additionally, GPS data may be received from a cellular phone through the transceiver 122, for example over a Bluetooth connection. In some embodiments, cellular triangulation may be used to measure the location of the sensor package 120.

The location data from location sensor(s) 123 may be associated with any of the outputs from the various sensors, e.g., images from imaging sensor(s) 130 and depth information depth sensor(s) 138, in memory 157 or through being simultaneously transmitted. The location data received from location sensor(s) 123 may be used by a server (e.g., 116) or any suitable processing circuitry to associate information related to the harvested specialty crops with the locations at which the crops were harvested. In some embodiments, the images from imaging sensor(s) 138 may be geotagged with location data from GPS (Global Positioning System) receiver 125.

In some embodiments, processing circuitry generates a map of the yield of harvested specialty crops using the location data. In some embodiments, the captured images and depth data are associated with location data, and the characteristics of the harvested specialty crops determined from the location and image data are also associated with the location data. The associations with location may be used to generate a map for display to a user. For example, in some embodiments, a map may associate GPS coordinates with the proportion of crops that meet a certain USDA grade.

In some embodiments, the location data from the location sensor(s) 123 may be cached and only transmitted when the specialty crops harvested at the particular location pass under the sensor package 120 in order to compensate for a pause in the conveyor carrying harvested specialty crops, for example when a truck pulls away from a harvester.

In some embodiments, the data collected by an IMU or one or more of a gyroscope, an accelerometer, and a magnetometer may be used to refine GPS-based location estimates. In some embodiments, the location sensor(s) 123 is in electrical communication with GPS pin input 155, which is used to indicate a location of interest to a user. In some embodiments, GPS pin input 155 is a button on the exterior of the sensor package 120 or otherwise accessible to an operator, e.g., through a wired or wireless connection to a cellphone, computer, or the cabin of a tractor or harvester. In response to receiving a signal on the GPS pin input 155, the processing circuitry 112 and the location sensor(s) 123 will store the current location data and indication, which may be referred to as a GPS pin, that the current location data relates to a location of interest in memory 157. The GPS pin input 155 may be useful for identifying locations in the field where rot or suboptimal moisture conditions are detected.

In some embodiments, an operator may provide input at a certain point in time (e.g., by pressing a button on the sensor package or an interface communicatively coupled to the sensor package) to provide an indication that an event of interest occurred at that time. This time-point may then be associated with the data collected by the sensor package and operate as a “note” or “pin” that can facilitate subsequent review of collated data. For example, a human user riding a harvester or tractor could place a pin in time and associate the pin with a certain condition. For example, the human user may place a pin whenever rotten potatoes pass under the belt of the harvester.

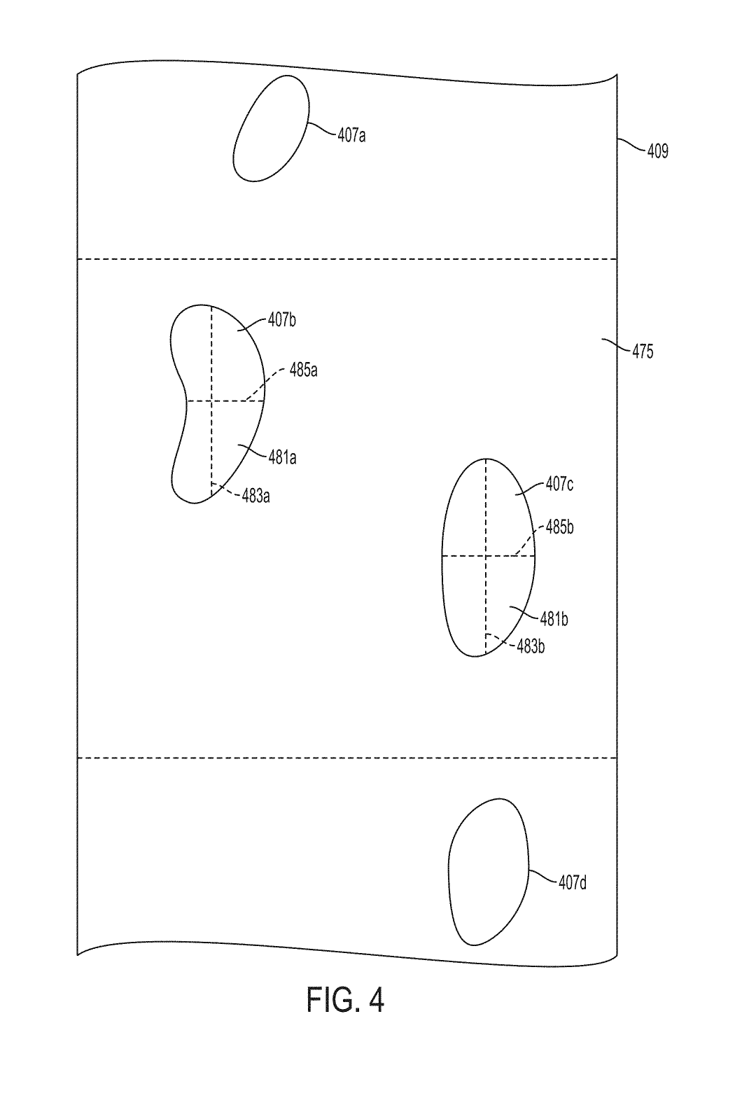

FIG. 4 shows an illustrative image of crops being measured for individual characteristics, in accordance with some embodiments of the technology described herein. FIG. 4 shows conveyor 409, harvested specialty crops 407a . . . 407d, and field of view 475. The conveyor 409 carries the harvested specialty crops 407a . . . 407d through the field of view 475. The harvested specialty crop 407a has already been observed by a sensor package. The harvested specialty crop 407d has not passed through the field of view 475 and has not yet been observed. The harvested specialty crops 407b and 407c are being measured for individual dimensions and geometric characteristics. When imaged, processing circuitry will analyze the image using edge detection techniques and clustering techniques to detect perimeter 481a of harvested specialty crop 407a and perimeter 481b of harvested specialty crop 407c. Using further image analysis, processing circuitry can detect major axes 483a and 483b and minor axes 485a and 485b, which can be used to calculate the volume of individual specialty crops, as described with reference to other aspects of the disclosure.

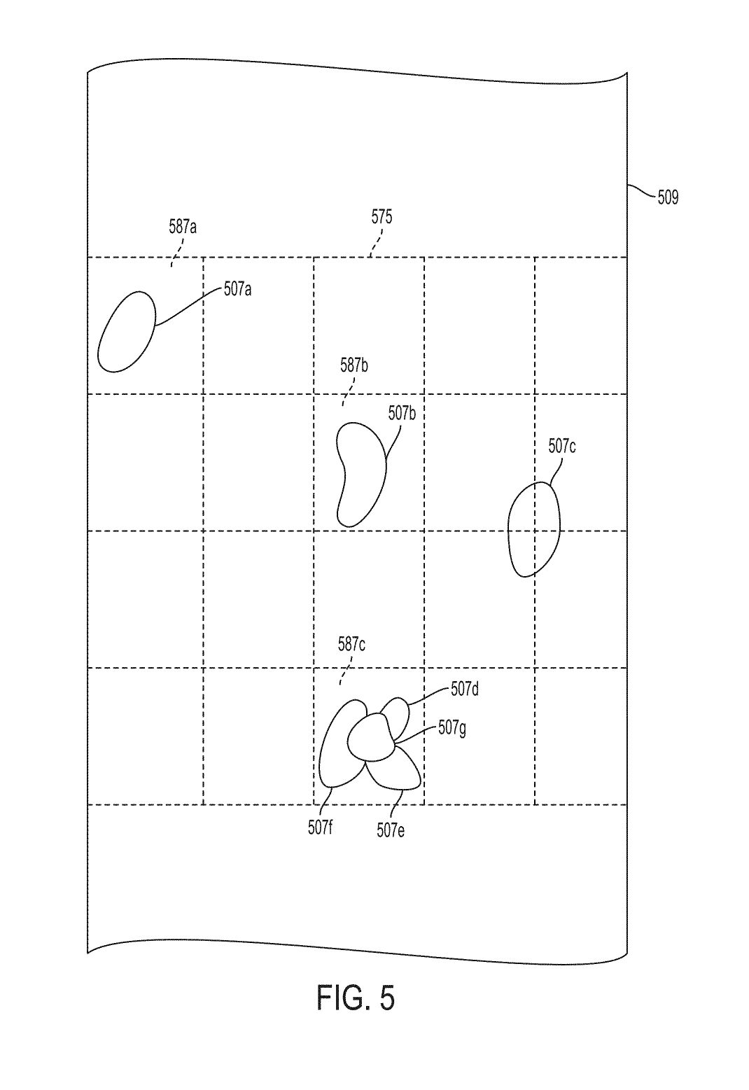

FIG. 5 shows an illustrative image of the depth of crops being measured, in accordance with some embodiments of the technology described herein. FIG. 5 includes conveyor 509, harvested specialty crops 507a . . . 507g, field of view 575, and grid sections 587a . . . 587c. The field of view 575 is divided into a virtual grid with multiple sections, such as the grid section 587a . . . 587c.

As described herein, in some embodiments, in order to reduce transmissions and/or bandwidth from a sensor package, the sensor package may compress the data collected by one or more of its sensors. For example, in some embodiments, the sensor package may compress depth information obtained from data gathered, at least in part, by using one or more depth sensors (and, in some embodiments, additionally using one or more imaging sensors). As described herein, computing the average depth in a section of a grid may substantially reduces the information required compared to, for example, a LIDAR point cloud or stereoscopic image without substantially inhibiting the accuracy of volume calculations. For example, as shown in FIG. 5, the grid sections 587a and 587b contains single harvested specialty crops 507a and 507b, respectively. The average depths in the sections will be less than the height, e.g., the minor axis diameter, of the harvested specialty crops 507a and 507b since the grid sections 587a and 587b contain areas of zero specialty crop depth, but the reported average depth will show the correct volume for each of the grid sections 587a and 587b. The harvested specialty crop 507c crosses the boundaries of multiple grid sections so each section will count a portion of the volume. The harvested specialty crops 507d . . . 507g are stacked in a roughly tetrahedral shape, and the average depth information for grid section 587c will appear substantially identical to average depth information for a cuboid with volume equivalent to the tetrahedron.

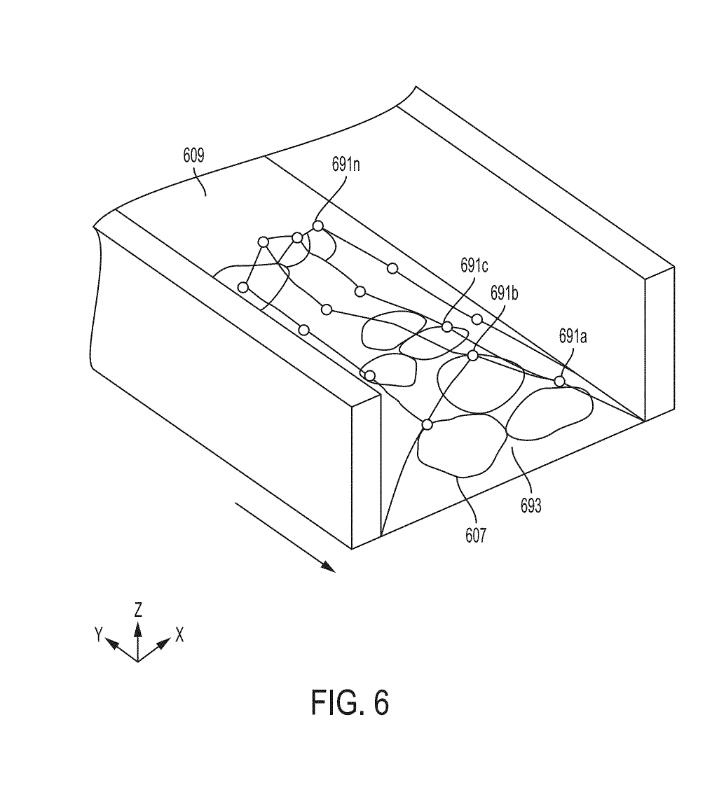

FIG. 6 illustrates a three-dimensional surface model of a set of harvested specialty crops, in accordance with some embodiments of the technology described herein. FIG. 6 includes conveyor 609, harvested specialty crops 607, points 691a . . . 691c and 691n included in surface mesh 693. In some embodiments, the measured depth information comprises a point cloud that is used to generate the mesh 693. The points 691a . . . 691n may be selected from the depth information in any suitable manner, for example being evenly spaced, representing the average of a nearby region of depth information, or to represent isoclines in the depth information. The surface mesh 693 may also include substantially all of the information in a LIDAR point cloud or stereoscopic image. In some embodiments, a volume integral taken of the surface mesh along the direction of crop movement may be used to estimate the volume of harvested specialty crops as well as a volumetric flow rate of the harvested specialty crops 607.

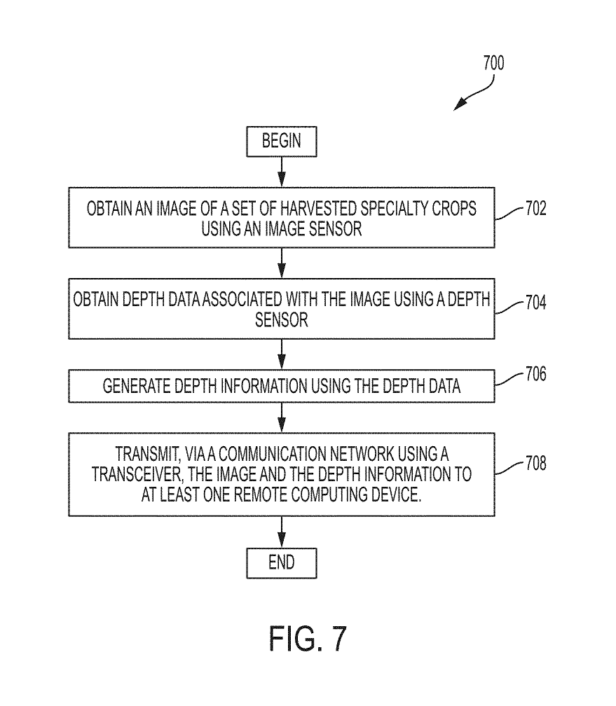

FIG. 7 is a flowchart of an illustrative process 700 for obtaining and transmitting data for monitoring and assessing characteristics of harvested specialty crops, in accordance with some embodiments of the technology described herein. Process 700 may be performed by any suitable device and, for example, may be performed by sensor package 111 described with reference to FIG. 1A.

As shown in FIG. 7, process 700 includes: (1) obtaining an image of harvested specialty crops at act 702, which may be done using an imaging sensor (e.g., imaging sensor 117); (2) obtaining depth data associated with the image at act 704 using a depth sensor (e.g., depth sensor 119); (3) generating depth information using the depth data at act 706; and (4) transmitting the image and the associated depth information, via at least one communication network, to a remote computing device at act 708.

At act 702, the sensor package performing process 700 obtains an image of a set of harvested specialty crops using an imaging sensor. Examples of imaging sensors are provided herein. As an illustrative non-limiting example, the image may be obtained by a camera mounted above a conveyor of a harvester or bin piler.

Next, process 700 proceeds to act 704, where the sensor package obtains depth data associated with the image using a depth sensor. Examples of depth sensors are provided herein. The depth data may include ultrasound data, LIDAR data, and/or imaging data. For example, the depth data may be a LIDAR point cloud, one or more pairs of stereoscopic images, a series of monoscopic images, or ultrasound data.

Next, at act 706, depth information may be generated using the depth data. The depth information may be derived from the depth data in any suitable way. For example, the depth data may indicate (directly, as in the case of LIDAR and ultrasound data, or after processing, as in the case of stereo image data) the distances to points on top surfaces of the crops. The depth information may then be obtained by subtracting these distances from the distance between the sensor package and a surface supporting the crops (e.g., a surface of the conveyor or storage bin). The distance between the sensor package and the surface supporting the crops may be determined in advance or measured when no crops are present on the surface.

Next, at step 708, the image and depth information are transmitted (e.g., using transmitter 121 or transceiver 122) via a communication network (e.g., network 114) to at least one remote computing device (e.g., the server 116) for subsequent processing and/or storage. As discussed herein, in some embodiments, the sensor package preprocesses information prior to transmission to the server in order to reduce the amount of information transmitted over the network, which may have limited bandwidth, since harvests may include tens of millions of individual specialty crops.

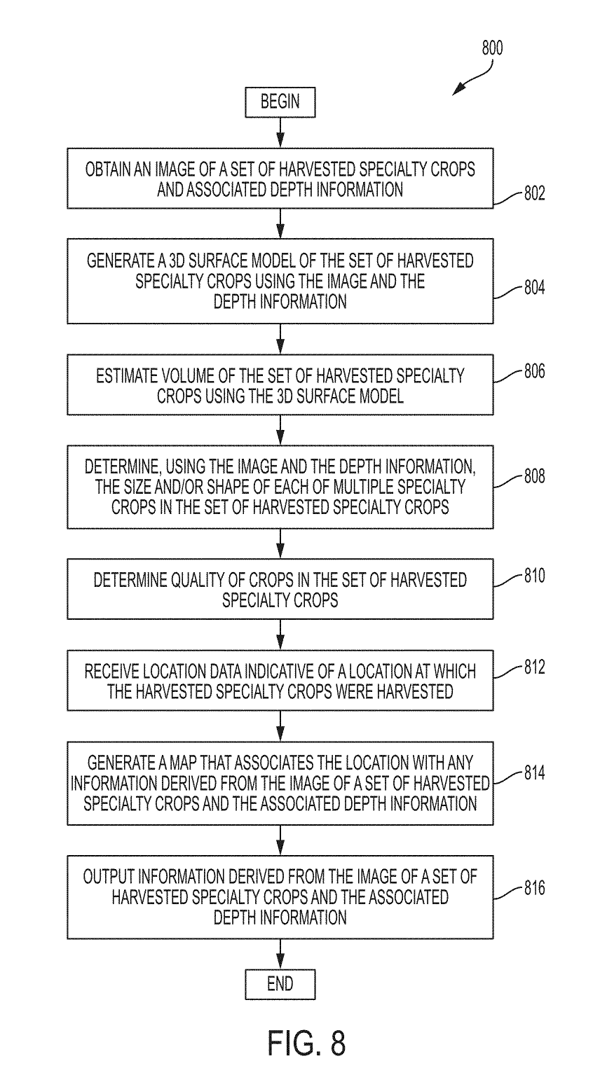

FIG. 8 is flow chart of an illustrative process 800 for monitoring and assessing characteristics of harvested specialty crops, in accordance with some embodiments of the technology described herein. Process 800 may be performed by any suitable computing device(s) and, for example, may be performed by server 116 described with reference to FIG. 1A.

As shown in FIG. 8, process 800 includes: (1) obtaining an image of a set harvested specialty crops and associated depth information at act 802; (2) generating a 3D surface model of the harvested specialty crops is generated at act 804; (3) estimating the volume of the set of harvested specialty crops at act 806; (4) determining the size and/or shape of each of multiple harvested specialty crops in the set of harvested specialty crops at act 808; (5) determining the quality of the harvested specialty crops at act 810; (6) receiving location data indicative of the location at which the harvested specialty crops were harvested at act step 812; (7) generating a map that associates the location data with any of the information derived at acts 804-812 regarding the harvested specialty crops at act 814; and (8) outputting any information derived as part of process 800 to a storage location, user, third party observer, and/or any other suitable destination at act 816.

It should be appreciated that, in some embodiments, one or more acts of process 800 may be omitted. For example, in some embodiments, only acts 802, 804, and 806 may be performed. As another example, in some embodiments, only acts 802 and 808 may be performed. As yet another example, in some embodiments, only acts 802 and 808 may be performed. Accordingly, it should be appreciated that, in some embodiments, one or more of acts 804-816 may be omitted.

Process 800 begins at act 802, where an image of harvested specialty crops and associated depth information are obtained. In some embodiments, these data may be obtained (e.g., in real time), via a communication network, from a sensor package. In some embodiments, these data may be accessed at a storage location (e.g., a memory accessible by the computing device(s) executing process 800) after being placed there at a previous time.

Next, at act 804, a 3D surface model of the set of harvested specialty crops is generated using the image and depth information received at act 802. This may be done in any suitable way. For example, in some embodiments, the depth information may include a grid of depth measurements, for example as discussed with reference to FIG. 5, in which case the 3D model can be generated as a collection of cuboids with cross sectional dimensions equal to the dimensions of each grid section, which may or may not be uniform, and heights equal to the average depth measurement in each grid section. In some embodiments, the 3D model may be generated as surface mesh, for example as discussed with reference to FIG. 6, in which points representing depth information are connected with vectors. The points in the mesh may be placed at regular intervals (e.g., at intersections on a grid), at locations selected based on the depth information, or with any suitable resolution. The 3D model may be updated over time as additional image and associated depth data are obtained.

In some embodiments, the computing device performing process 800 may determine features, such as patterns of pixel values, that are common to multiple images of the harvested set of specialty crops and use the common features to determine an offset distance representing the distance the harvested specialty crops moved in-between the capture of the images. In some embodiments, the offset distance may be calculated using information received from the sensor package or calculated by the processing circuitry to represent the speed of the specialty crop conveyor (e.g., conveyor 09). The offset distance may be used to avoid double counting any of the harvested specialty crops and may stitch together multiple images or supplement the 3D surface model with the new harvested specialty crops. In some embodiments, the series of images do not overlap and statistical inference is used to estimate the overall crop yield using data related to each image and/or depth measurement as a sample.

Next, at act 806, the volume of the set of harvested specialty crops may be estimated using the 3D surface model generated at act 804. In some embodiments, the volume may be determined by computing a volume integral of the area underneath the 3D surface model. In some embodiments, e.g., where the surface model includes a collection of cuboids, the volume may be calculated by calculating the volume of portions of the surface model, e.g., one cuboid or section of the mesh at a time, and sum the volumes of the portions. In some embodiments, the processing circuitry will sum portions that are perpendicular to the direction of the flow of crops on the conveyor and pass under the sensor package at the same time in order to account for crops that were harvested in substantially the same location or at the same time for association with sensor data.

In some embodiments, the computing device(s) performing process 800 may access a database to determine the density of the given specialty crops being processed, which may be identified by a harvester operator, a human observer, image analysis, or any suitable means. The density of the harvested specialty crops and the volume may be used to determine the mass of harvested specialty crops. In addition, the computing device(s) may estimate, based on the received images, a portion of the flow of material that is debris and deduct the estimated volume of debris from the 3D surface model volume measurements to provide a more accurate volume estimate.

In some embodiments, the techniques described herein may be used to estimate the portion or percentage of tare in the flow of material. Such estimates may be combined with weight measurements provided by conventional load cell/weight-based systems in order to correct the weight estimates provided by these systems thereby improving the functionality of such systems.

Next, at step 808, the image and depth information are used to determine the size and/or shape of each of multiple specialty crops in the set of harvested specialty crops. For example as discussed with reference to FIG. 4, edge detection may be performed on the image detect the edges of the harvested specialty crops. Subsequently clustering may be applied to the detected edges to create edge groups associated with individual harvested specialty crops. Lengths of major and minor axes of each specialty crop may be determined from the edge groups. These lengths may be used to estimate the volume each individual specialty crop.

Next, at act 810, the quality of crops in the set of harvested specialty crops may be determined. In some embodiments, one or more geometric characteristics of an individual crop (e.g., volume, length, diameter, length or major axis, length of minor axis, etc.) may be used to estimate the quality of the crop. For example, the lengths of the major and minor axes may be used to determine whether an individual potato is graded as a “USDA No. 1” potato or a “USDA No. 2” potato.

In some embodiments, the image obtained at act 802 may be provided, as part of act 810, as input to a trained statistical model and the output of the trained statistical model may provide an indication of the quality of the individual specialty crop. For example, the output of the trained statistical model may indicate whether the individual specialty crop has rot, bruising, discoloration and/or any other characteristic indicative of its quality. Examples of quality characteristics and different types of trained statistical models are provided herein.

Next, at act 812, location data indicative of a location at which the harvested specialty crops were harvested may be obtained. In some embodiments, the location data may be received, via a communication network, from a sensor package coupled to a harvester (e.g., from the same sensor package that obtained the image and the associated depth data at act 802). In some embodiments, the location data may provide an indication of where in a storage facility crops in the image are stored. In such embodiments, the location data may be received from a sensor package coupled to a bin piler in a storage facility rather than a harvester in a field.

In some embodiments, the location data may have been received previously and stored, and may be accessed at act 812. The location data may be GPS data, corrected GPS data (e.g., corrected based on one or more IMU measurements), coordinate data, position data, and/or any other suitable data indicating a location at which the crops in the image obtained at act 802 were harvested.

Next, at act 814, a map is generated that associates the location data with any information derived as part process 800. For example, the location data may be associated with crop volume, mass, quality and/or any other suitable information. The map may be stored for subsequent use and/or provided to one or more human users (e.g., a coordinator of harvesting in a field, one or more people working in a storage facility, etc.)

Next, at step 816, any of the information derived during process 800 from the image of the set of harvested specialty crops and the associated depth information is output. In some embodiments, the information may be stored for subsequent use. In some embodiments, the information may be provided to an operator of harvesting equipment (e.g., a harvester or bin piler) and the operator may alter the operation of the harvesting equipment based on the received information. Examples of this are provided herein. In some embodiments, the information may be used to automatically control operation of the harvesting equipment instead of being provided to a human user. In some embodiments, the information may be used to determine a price that a grower is to be paid for the harvested specialty crops.

Aspects of the technology described herein may provide one or more benefits, some of which have been previously described. Now described are some non-limiting examples of such benefits. It should be appreciated that not all aspects and embodiments necessarily provide all of the benefits now described. Further, it should be appreciated that aspects of the technology described herein may provide additional benefits to those now described.

Aspects of the technology described herein provide a system for monitoring and assessing characteristics of harvested specialty crops. The system includes one or more sensor packages configured to gather data about harvested specialty crops and may be programmed with one or more novel algorithms that process the gathered data to estimate volume, mass, and quality of the harvested crops (on an individual and aggregate basis). The sensor packages are portable, ruggedized, and robust such that they may operate in field environments (e.g., when coupled to a harvester, a tractor, a pick-up truck) as well as in pack houses, and processing and storage facilities. Data collected by the system may include geo-temporal information allowing precise mapping of harvested crops to indicate where and when they were harvested in the field, as well as with respect to locations in a storage facility indicating where and when in the storage facility the harvested crops were stored.

In some embodiments, the data gathered by the system may be used to alter (e.g., optimize) the harvesting process in real-time, for example by raising/lowering harvesting implements, stopping harvesting, and the like. In some embodiments, the system provides a machine learning infrastructure for generating training data (e.g., by allowing crowd-sourcing based labeling of crop images) to generate trained statistical models that may be used for determining crop quality for individual crops and in the aggregate. The system is extensible and may be easily upgraded, for example, to allow for handling new crop varieties.

The above-described embodiments of the technology described herein may be implemented in any of numerous ways. For example, the embodiments may be implemented using hardware, software, or a combination of hardware and software. When implemented in software, the software code may be executed on any suitable processor or collection of processors, whether provided in a single computer or distributed among multiple computers.

Such computers may be interconnected by one or more communication media (e.g., networks) in any suitable form, including a local area network (LAN) or a wide area network (WAN), such as an enterprise network, an intelligent network (IN) or the Internet. Such networks may be based on any suitable technology and may operate according to any suitable protocol and may include wireless networks, wired networks, and/or fiber optic networks. Such network(s) may be an intelligent, interconnected network which may facilitate provisioning of accurate harvest estimates by the United States Department of Agriculture, provide data to industry consortia and/or other groups, among other benefits.

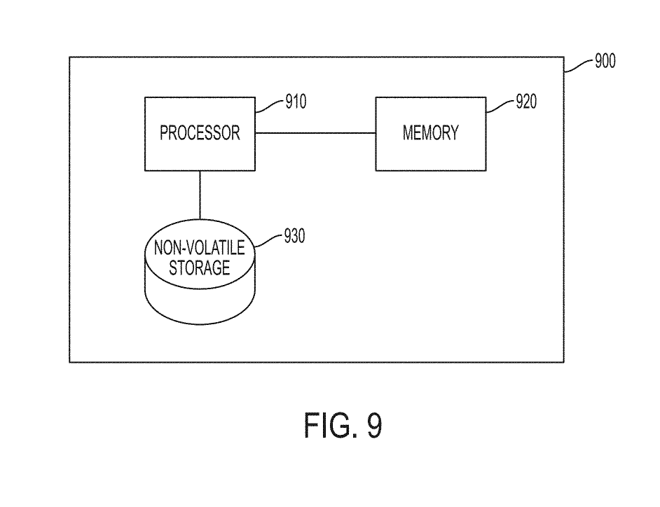

An illustrative implementation of a computer system 910 that may be used in connection with any of the embodiments of the technology described herein is shown in FIG. 9. The computer system 900 may include one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited in this respect. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the technology described herein need not reside on a single computer or processor, but may be distributed in a modular fashion among different computers or processors to implement various aspects of the present invention.

Processor-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more methods, of which examples have been provided, including with reference to FIGS. 7 and 8. The acts performed as part of each method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

What is claimed is:

1. A system for use in connection with assessing characteristics of harvested specialty crops, the system comprising:
at least one computer hardware processor; and
at least one non-transitory computer-readable storage medium storing processor-executable instructions that, when executed by the at least one computer hardware processor, cause the at least one computer hardware processor to perform:
obtaining an image of a set of harvested specialty crops and associated depth information;
generating a 3D surface model of the set of harvested specialty crops using the image and the depth information; and
estimating the volume of the set of harvested specialty crops using the 3D surface model.

2. The system of claim 1, wherein the 3D surface model comprises a 3D mesh.

3. The system of claim 1, wherein estimating the volume of the set of harvested specialty crops comprises computing a volume integral using the 3D surface model.

4. The system of claim 3, wherein the 3D surface model comprises a plurality of sections, and wherein computing the volume integral comprises:
estimating, using dimensions and depth information for each section of the 3D surface model, a sectional volume of harvested specialty crops in each section, to obtain a plurality of sectional volumes; and
computing a sum of the plurality of sectional volumes.

5. The system of claim 1, wherein the processor-executable instructions, when executed by the at least one computer hardware processor, further cause the at least one computer hardware processor to perform:
accessing, in a database, a density for a type of specialty crop in the set of harvested specialty crops; and
estimating a mass of the set of harvested specialty crops using the volume of the set of the harvested specialty crops and the density.

6. The system of claim 1, wherein the processor-executable instructions, when executed by the at least one computer hardware processor, cause the at least one computer hardware processor to perform:
obtaining a second image of a second set of harvested specialty crops and associated second depth information;
generating a second 3D surface model of the second set of harvested specialty crops using the second image and the second depth information;
estimating the volume of the second set of harvested specialty crops using the second 3D surface model; and
adding the estimated volume of the second set of harvested specialty crops to the estimated volume of the set of harvested specialty crops.

7. The system of claim 1, wherein the processor-executable instructions, when executed by the at least one computer hardware processor, cause the at least one computer hardware processor to perform:
receiving location data indicative of a location at which the harvested specialty crops were harvested; and
generating a map that associates the location with any information derived from the image of a set of harvested specialty crops and the associated depth information.

8. A system for use in connection with assessing characteristics of harvested specialty crops, the system comprising:
at least one computer hardware processor;
at least one non-transitory computer-readable storage medium storing processor-executable instructions that, when executed by the at least one computer hardware processor, cause the at least one computer hardware processor to perform:
obtaining an image of a set of harvested specialty crops and associated depth information; and
determining, using the image and the depth information, the size and/or shape of each of multiple specialty crops in the set of harvested specialty crops.

9. The system of claim 8, wherein the determining comprises:
applying an image edge detection technique to the image to obtain detected edges;
identifying, using the detected edges, boundaries of a first harvested specialty crop in the set of harvested specialty crops; and
determining, using the identified boundaries, a length of a major axis of the first harvested specialty crop and a length of a minor axis of the first harvested specialty crop.

10. The system of claim 9, wherein the processor-executable instructions, when executed by the at least one computer hardware processor, cause the at least one computer hardware processor to perform:
computing, using the length of the major axis distance and the length of the minor axis, a volume and/or a surface area of the first harvested specialty crop.

11. The system of claim 8, wherein the processor-executable instructions, when executed by the at least one computer hardware processor, cause the at least one computer hardware processor to perform:
generating a 3D surface model of the set of harvested specialty crops using the image and the depth information; and
estimating the volume of the set of harvested specialty crops using the 3D surface model.

12. The system of claim 9, wherein the processor-executable instructions, when executed by the at least one computer hardware processor, cause the at least one computer hardware processor to perform:
accessing a trained statistical model configured to output information indicative of harvested specialty crop quality;
providing, as input to the trained statistical model, at least one feature selected from the group consisting of the image, the depth information, the length of the major axis, and the length of the minor axis; and
determining quality of crops in the set of harvested specialty crops based on output of the trained statistical model.

13. The system of claim 12, wherein the trained statistical model comprises a trained convolutional neural network.

14. A method for use in connection with assessing characteristics of harvested specialty crops, the method comprising:
using at least one computer hardware processor to perform:
obtaining an image of a set of harvested specialty crops and associated depth information; and
determining, using the image and the depth information, the size and/or shape of each of multiple specialty crops in the set of harvested specialty crops.

15. The method of claim 14, wherein the determining comprises:
applying an image edge detection technique to the image to obtain detected edges;
identifying, using the detected edges, boundaries of a first harvested specialty crop in the set of harvested specialty crops; and
determining, using the identified boundaries, a length of a major axis of the first harvested specialty crop and a length of a minor axis of the first harvested specialty crop.

16. The method of claim 15, further comprising:
computing, using the length of the major axis distance and the length of the minor axis, a volume and/or a surface area of the first harvested specialty crop.

17. The method of claim 14, further comprising:
generating a 3D surface model of the set of harvested specialty crops using the image and the depth information; and
estimating the volume of the set of harvested specialty crops using the 3D surface model.

18. The method of claim 15, further comprising:
accessing a trained statistical model configured to output information indicative of harvested specialty crop quality;
providing, as input to the trained statistical model, at least one feature selected from the group consisting of the image, the depth information, the length of the major axis, and the length of the minor axis; and
determining quality of crops in the set of harvested specialty crops based on output of the trained statistical model.

19. The method of claim 18, wherein the trained statistical model comprises a trained convolutional neural network.

20. The method of claim 18, wherein the trained statistical model comprises a trained Bayesian classifier.
